(19) United States
(12) Patent Application Publication
(10) Pub. No.: US 2024/0055101 A1
(43) Pub. Date: Feb. 15, 2024

(54) FOOD AND NUTRIENT ESTIMATION, DIETARY ASSESSMENT, EVALUATION, PREDICTION AND MANAGEMENT

(71) Applicants: Trustees of Tufts College, Medford, MA (US); Research Foundation of the City University of New York, New York, NY (US)

(72) Inventors: Karen A. Panetta, Rockport, MA (US); Shishir Paramathma Rao, Burlington, MA (US); Shreyas Kamath Kalasa Mohandas, Burlington, MA (US); Rahul Rajendran, Belleville, MI (US); Srijith Rajeev, Burlington, MA (US); Erin Hennessy, North Reading, MA (US); Christina Economos, Concord, MA (US); Eleanor Tate Shonkoff, Reading, MA (US); Sos S. Agaian, New York, NY (US)

(73) Assignees: Trustees of Tufts College, Medford, MA (US); Research Foundation of the City University of New York, New York, NY (US)

(21) Appl. No.: 18/256,971

(22) PCT Filed: Dec. 17, 2021
(86) PCT No.: PCT/US2021/063994

§ 371 Date: Jun. 12, 2023

Related U.S. Application Data
(60) Provisional application No. 63/127,119, filed on Dec. 17, 2020.

(51) Int. Cl. G16H 20/60; G16H 50/20; G06Q 10/06; G06N 3/0464; G06N 3/08; G06N 20/10

(52) U.S. Cl. CPC G16H 20/60; G16H 50/20; G06Q 10/06; G06N 3/0464; G06N 3/08; G06N 20/10

(57) ABSTRACT

The disclosure generally relates to the artificial intelligence (AI) automatic methods, computer program product, and systems and methodology for dietary and medical treatment planning, food waste estimation, analyzing three-dimensional food image construction, measurement, nutrient estimation, nutritional assessment, evaluation, prediction and management. More particularly, the embodiments described herein relate to utilizing an AI-based algorithm that can automatically, detect food items from images acquired by cameras for dietary assessment, dietary planning, and for estimating food waste. In one aspect, the method may include food calorie estimation techniques using machine learning and computer vision techniques for dietary assessment. In another aspect, the tools may apply to personalized nutrition. The method may also include the automation of nutrition planning. In yet another aspect, the tools may apply to medical treatment planning, wherein meals and treatment plans are individualized explicitly for each user according to several unique characteristics associated with that user.

### CROSS-REFERENCE TO RELATED APPLICATIONS

The present application claims the benefit of U.S. Provisional Patent Application No. 63/127,119, filed on Dec. 17, 2020, which is incorporated herein by reference.

### STATEMENT REGARDING FEDERALLY SPONSORED RESEARCH

This invention was made with Government support under Grant No. CA250024 awarded by the National Institutes of Health. The Government has certain rights in the invention.

### BACKGROUND

Over the years, increased light has been shed on an individual's nutrient assessment and food waste reduction along the food supply chain (FSC). These efforts directly relate to human health and the country's sustainable development, thus attracting great social concern and global attention. Recent studies by the Centers for Disease Control and Presentation have indicated that in 2015-16 alone, the prevalence of obesity was 39.8% and affected about 93.3 million U.S. adults (https://www.cdc.gov/obesity/data/adult.html). Another report from Trust for America's Health and the Robert Wood Johnson Foundation estimates obesity rates to reach 44 percent by 2030. Diabetes, coronary heart disease, stroke, cancer, and osteoarthritis are some of the illnesses associated with obesity that impose human suffering as well as significant medical costs (Wang, Y. Claire, et al. “Health and economic burden of the projected obesity trends in the USA and the UK.” The Lancet 378.9793 (2011): 815-825). National Health and Nutrition Examination Survey (NHANES), a program of studies designed to assess the health and nutritional status of adults and children in the United States, found that individuals with low incomes are more likely to be affected by obesity when compared to individuals with high incomes (Ogden, Cynthia L., et al. “Obesity and Socioeconomic Status in Children and Adolescents: the United States, 2005-2008. NCHS Data Brief. Number 51.” National Center for Health Statistics (2010)). Major contributing factors to the disproportional impact of obesity on populations from lower-income backgrounds in America include the barriers faced by people living in poverty in accessing healthy foods, a lack of nutrition education, a dearth of safe environments for physical activity, and recreation, and food marketing targeted to this population.

According to Food Allergy Research and Education (FARE) research, 15 million Americans have food allergies, including 5.9 million children under age 18. Among them, 30 percent of the children are allergic to more than one food item. Major food allergens include milk, egg, peanut, tree nuts (for example, walnuts, almonds, cashews, pistachios, pecans), wheat, soy, fish and crustacean shellfish in the USA (http://www.webster.edu/specialevents/planning/food-information.html). These allergens are generally consumed as a subset of the ingredients of food items. Due to this, the majority of these individuals are on restricted diets. Another restriction is due to regulated health conditions, such as diabetes, high cholesterol, gout, high blood pressure, and celiac disease. Managing a restricted diet is a challenging task for individuals as diet varies for different individuals. For example, a family with multiple members may have dietary restrictions based on specific allergies of individual members. Another concern is that each allergen has multiple classes with subclasses. the main class may cause allergy in a few cases while the subclass may not, resulting in additional dietary constraints.

Another mounting problem in the United States and elsewhere is food waste (J. Buzby, H. Farah-Wells, and J. Hyman, “The estimated amount, value, and calories of postharvest food losses at the retail and consumer levels in the United States,” 2014). According to a USDA's Economic Research Service 2010 report, 133 billion pounds of the 430 billion pounds of the national food supply went uneaten. Based on average retail prices, this was equated to about $161.6 billion. One of the major causes of food waste is a lack of regard for the far-reaching effects of food waste and a poor understanding of the true value of food. Another cause is a lack of meal planning that provides more accurate estimates of the food a person actually needs to buy and consume. Unnecessary impulse and bulk purchases also add to the food waste problem. Natural Resources Defense Council cites over-preparation and spoilage as reasons that contribute significantly to food waste.

Accurate approaches and tools to evaluate the aforementioned problems are essential in monitoring the nutritional status of individuals for epidemiological and clinical research on the association between diet and health. Traditional methods require time-consuming manual nutrition coding and are expensive, especially when methods such as 24-h dietary recall (24 HR) interviews, and food record (FR) are involved (M. C. Carter et al., “Development of a UK online 24-h dietary assessment tool: myfood24,” Nutrients, vol. 7, no. 6, pp. 4016-4032, 2015). Food Frequency Questionnaires (FFQ) are more affordable but are subject to measurement error. While significant advancements in nutrition and health fields have been made, reliable, accurate assessment of dietary intake remains elusive for this field (F. Zhu, M. Bosch, C. J. Boushey, and E. J. Delp, “An image analysis system for dietary assessment and evaluation,” in Image Processing (ICIP), 2010 17th IEEE International Conference on, 2010, pp. 1853-1856: IEEE).

Even though recall of foods/beverages consumed is an easy task for humans, volume/calorie estimation is not. Researchers have tried to address this problem with various computer vision techniques. For example, a technique described by Nakao, Koji. in “Portable terminal, calorie estimation method, and calorie estimation program.” U.S. patent application Ser. No. 13/305,012, developed a portable terminal used to capture images and estimate calories based on container shape, container color, and food color. Divakaran, Ajay, et al in “Automated food recognition and nutritional estimation with a personal mobile electronic device.” U.S. Pat. No. 9,734,426. 15 Aug. 2017, developed a methodology that recognized food items and provided nutritional estimation using a personal mobile electronic device. This system used multi-scale feature extraction to detect and classify food items and provide nutritional information based on the estimated portion size. Connor, Robert A. “Caloric intake measuring system using spectroscopic and 3D imaging analysis.” U.S. Pat. No. 9,442,100. 13 Sep. 2016, proposed yet another technique that uses the spectroscopic sensor to estimate food composition using light that is absorbed by or reflected from food and an imaging device that determines the quantity of the food. Sze, Calvin Lui, et al. “Nutrition intake tracker.” U.S. Pat. No. 7,432,454. 7 Oct. 2008 utilized a radio frequency identification (RFID) tag with nutrition information for each kind of food at various places. The food plate is placed on a coaster with an RFID reader and miniature built-in scale. The scale is used to measure the weight of a particular food placed on the plate. Tamrakar, Amir, et al. “Method for computing food volume in a method for analyzing food.” U.S. Pat. No. 8,345,930. 1 Jan. 2013, introduced a computer-implemented methodology that uses two sets of the plurality of images with different angular spacing per set. A 3-D point cloud is reconstructed based on the rectified pair of images among those sets and volume is estimated using those surfaces. Several commercial software applications have also been introduced, such as CalorieKing, MyFitnessPal, and Lose It!. Most of these software solutions require manual data entry, which leads to a poor estimation of calories. Moreover, it is tedious and time-consuming. Therefore, several methodological aspects of food and nutrient consumption in uncertain conditions still need further improvement to ensure reliable and valid estimation of dietary intake for decision making.

In terms of managing and substituting allergens with alternatives, it is an arduous task that has not been completely resolved by existing approaches (often static and standardized). Several organizations have set up regulations to overcome these issues up to some extent. In United States, according to the Food Allergen Labeling and Consumer Protection Act (FALCPA, Food Allergen Labeling and Consumer Protection Act of 2004, 21 U.S.C. 301), the law requires that every processed food item should contain a label that identifies food source names of all the ingredients along with major allergens or derivatives of these allergens mentioned previously must be declared in that label. Similarly, Food Standards Australia New Zealand has this code and has added lupin to the list of allergens since 25 May 2017. These approaches are well-intentioned; however, they are available only on processed food packages. In restaurant settings, even though the allergens can be mentioned to the chef beforehand, there might be several cases wherein the chef might unintentionally use subclasses of those allergens. Today, food labels and recipes have been digitized and are available over different digital media. Allrecipes.com (http://www.allrecipes.com), Yummly (http://www.yummly.com/), and Fooducate (http://www.fooducate.com) are but a few examples of this migration from paper to electronic access. The benefits are universal access without the need for a plethora of physical paper products nearby and ready access to expanded and new instances of the subject matters. Formats have emerged to represent the different components of a recipe. They include hRecipe, a simple, open, distributed format, suitable for embedding information about recipes for cooking in (X)HTML, Atom, RSS, and arbitrary XML (http://microformats.org/wiki/hrecipe), and RecipeML (http://www.formatdata.com/recipeml/spec/recipeml-spec.html). Even though the electronic form of nutrient information is available, current diet/nutrition planning tools are just used as a basis for calorie computation. These tools do not consider the relationships between genes and food, and the effect of food on the body.

Briancon, Alain Charles, et al. “Presentation of food information on a personal and selective dynamic basis and associated services.” U.S. patent application Ser. No. 14/259,837, presented a food media processing platform (FMPP) that processes food nutrition information for presentation to a consumer. Modified food nutrition information was generated based on the contrast between original food nutrition information stored in a first database and consumer provided information stored in a second database. An algorithm was developed that considers the critical attributes in the modified food nutrition information and presented to the consumer along with supplemental information. Mosher, Michele. “System and method for automated dietary planning.” U.S. patent application Ser. No. 11/069,096, filed yet another methodology, which provides meals and treatment plans specific to a user based on unique characteristics associated with that user. Brier, John. “Apparatus and method for providing on-line customized nutrition, fitness, and lifestyle plans based upon a user profile and goals.” U.S. patent application Ser. No. 10/135,229, presented a technique for generating customized wellness plans tailored to the individual. This system used an individual's answer for a specific set of questionnaires and generated personalized plans that include a nutrition plan, a fitness or work-out plan, and a lifestyle plan such as stress-reduction activities.

Steps for these non-invasive food volume/calorie estimation systems are food recognition, 3-D reconstruction, volume estimation, and mapping estimated volume to nutrient information. Food recognition from images is challenging as food types and items vary depending on demographic regions. Moreover, a single category/type of food may have significant variations. Most of the state-of-the-art techniques are designed for ideal/controlled laboratory conditions, which include a) well-separated food items and b) limited classes of food items. This aids in successful feature extraction but fail during classification due to a large number of food classes.

Moreover, in uncertain conditions with varying illumination and images with low resolution, blurring, and cluttered background, they perform poorly (Pouladzadeh, Parisa, Abdulsalam Yassine, and Shervin Shirmohammadi. “Food: food detection dataset for calorie measurement using food images.” International Conference on Image Analysis and Processing. Springer, Cham, 2015). Another major problem is the scarcity of publicly available food image datasets, which makes comparison/training of food recognition methods more arduous.

Accurate reconstruction of a 3-D model is useful to estimate the volume and weight. To perform a 3-D reconstruction, multiple images are required with the right amount of visible overlap of physical points. Corresponding points from these images are used to find 3-D coordinates of the points and construct a model. Several ways exist to reconstruct 3-D models using 2-D images such as laser scanning, stereo vision (using two cameras), structured light (one camera and one projector). While these methods provide various options to generate 3-D representation according to their needs, each method has limitations to some degree, such as costly instrument, limited operations, and/or working in a dark environment. These techniques can perceive depth directly from 2-D images. However, they require specific hardware to obtain a 3-D model of the food. Before capturing 2-D images using these techniques, camera calibration is a must. Once, camera calibration is satisfied, finding corresponding projections (finding the same point from two different cameras) is a difficult task. Moreover, adopting these techniques to various available cameras is challenging.

Food portion estimation (volume estimation) is an important task to accurately estimate nutrient information—this aids in obtaining the volume of the food item in consideration and calculate the nutrition content of the food consumed or even amount of food wasted. The images captured are two-dimensional and do not have depth information. Even though methods exist to estimate weight using digital images (E. A. Akpro Hippocrate, H. Suwa, Y. Arakawa, and K. Yasumoto, “Food weight estimation using smartphone and cutlery,” in Proceedings of the First Workshop on IoT-enabled Healthcare and Wellness Technologies and Systems, 2016, pp. 9-14: ACM, B. Zhou et al., “Smart table surface: A novel approach to pervasive dining monitoring,” in Pervasive Computing and Communications (PerCom), 2015 IEEE International Conference on, 2015, pp. 155-162: IEEE), they are unreliable as they are non-real world estimates. The state-of-the-art techniques either require a template (such as spoons, tablecloth, markers) to construct 3-D model or assume the food items to have a specific shape (J. Dehais, M. Anthimopoulos, S. Shevchik, and S. Mougiakakou, “Two-View 3D reconstruction for food volume estimation,” IEEE transactions on multimedia, vol. 19, no. 5, pp. 1090-1099, 2017). However, such templates are unavailable in a real-world scenario, and making such assumptions is unrealistic. Furthermore, the published works provide limited information on algorithmic choices and tuning, and most systems fail in a mixed food situation as they are developed only for ideal conditions.

Current existing technologies deal with an individual's diet planning/suggestions and individual's calorie/nutrient assessment separately. Moreover, most of them utilize traditional techniques with general rules, diets, and diet plans without considering individuals' requirements.

### SUMMARY

Example embodiments of the disclosure provide methods and apparatus for automated individual dietary planning incorporated with a dietary assessment. Embodiments may include a system that includes computer vision techniques in combination with artificial intelligence methods such as machine learning, deep learning, and/or neural networks (NN).

In one aspect, example embodiments of the disclosure provide an artificial intelligence-based method to generate and/or recommend meal plans, including recipes for dieters. This considers various data, such as body composition, weight fluctuation trends, and individual goals.

Some embodiments may generate and/or recommend personalized meal plans in which a multitude of dieter characteristics, such as food preferences, genetic characteristic, calorie/nutrient requirements, budget, and food allergies, are considered. This can also be combined with exercise, medical or drug treatment, and therapy.

In other embodiments, a system may provide a nutritional, supplement, and/or medical treatment therapy diet plan (for weight loss, chemotherapy, or other medical conditions) for allowing the individual to input various data, such as water and food consumption during therapy to track progress, allow other professionals such as, trainers, doctors, nutritionists to interact with the patient's therapy and record, and track and report on the progress of the therapy.

In embodiments, a system may provide an automated system for diet planning which operates to selectively purchase the food recommended within a menu plan. It may further comprise assisting individuals to buy food items according to their daily needs.

Example embodiments may provide an automated system for notifying the individuals about the freshness of the food items of purchased food items. It may further comprise assisting the individuals to recommend meal plans according to the freshness or the food expiration date.

Other embodiments may provide a set of building blocks for machine learning methodologies, including hypercomplex-based networks and/or alpha-trimmed-based networks arranged in any directed or undirected graph structure. This can be combined with any other types of network elements, including, for example, pooling, dropout, upsampling, and fully-connected traditional or hypercomplex neural network layers.

In one aspect, the present disclosure provides a method for dietary assessment that incorporates multimedia analytics, including capturing a plurality of 2-D images taken from different positions above the food plate with any image capturing device before consumption and after consumption; selecting food item after consumption, perform segmentation and detection of the said food items, reconstructing three-dimensional (3-D) images using a plurality of the said two-dimensional (2-D) images, computing volume using the 3-D image, mapping the volume to weight and estimating nutrition content in the food item. In some aspects, without limiting the scope of the present disclosure, the systems and methods discussed may be used with visible, near-visible, grayscale, color, thermal, computed tomography, magnetic resonance imaging, as well as video processing and measurement.

In one aspect, the present disclosure provides a method for classifying an acquired multimedia input. The method includes receiving the input multimedia content, applying a feature-based classification method of different food types to train a plurality of classifiers to recognize individual food items. Feature-based learning method may further comprise: selecting at least one or more images from the plurality of images from the same scene; processing these images; utilizing conventional techniques (for example, Scale-invariant feature transform (SIFT), edge, color, shape, corner, blob, ridge-based detectors) and/or machine learning-based techniques (for example convolutional neural networks, capsule networks, hypercomplex convolutions, alpha trimmed convolutions) to extract high dimensional image-based features; training a neural network to provide to propose the region of interest and identify each food type along with a confidence score; applying the trained classifier to new samples to validate the model, wrongly classified samples are added as new samples and the model is retrained; and stopping the training until convergence or incorrectly classified samples in the training images falls below a predetermined threshold.

In one aspect, the present disclosure provides a method for segmenting an acquired multimedia input. The method includes receiving the input multimedia content, applying a feature-based segmentation of different food types to train a plurality of classifiers to recognize individual food items. Feature-based learning method may further comprise: selecting at least one or more images from the plurality of images from the same scene; processing these images; utilizing the aforementioned high dimensional image-based features; training a neural network to provide generate masks for each food type; applying the trained segmentation methodology to new samples to validate the model, wrongly segmented samples are added as new samples and the model is retrained; and stopping the training until convergence or wrongly segmented samples in the training images falls below a predetermined threshold.

In one aspect, the present disclosure provides a method for three-dimensional image reconstruction may further comprise: capturing a plurality of 2-D images taken from different positions above the food plate with any image capturing device; extracting and matching multiple feature points in each image frame estimating relative camera poses among the plurality of 2-D images using the matched feature points; refining the correspondence until the best features are obtained; compute uncalibrated camera position and orientation calculation and 3-D structure estimation; perform camera self-calibration and scene calibration, generate a 3-D point cloud and densely reconstruct the obtained 3-D point cloud and perform texture mapping.

In yet another aspect, the present disclosure provides a method for three-dimensional image reconstruction using machine learning methods may further comprise: capturing a plurality of 2-D images taken from different positions above the food plate with any image capturing device; extracting high dimensional feature utilizing machine learning-based techniques (for example convolutional neural networks, capsule networks, hypercomplex convolutions, alpha trimmed convolutions), fusing the said features to obtain a sparse depth map; enhance the sparse depth to dense depth map using the aforementioned machine learning techniques and generate a 3-D point cloud.

In one aspect, the present disclosure provides a method for estimating the volume of the classified and segmented food item may further comprise: selecting at least one densely reconstructed 3-D food item with texture, dividing the 3-D food item into equal proportions/slices, computing volume of each slice and finally summing individual volumes of the said food item.

### BRIEF DESCRIPTION OF THE DRAWINGS

The present disclosure will hereafter be described with reference to the accompanying drawings, wherein like reference numerals denote like elements.

FIG. 1 is a flowchart illustrating the components of the system of the present disclosure in a preferred embodiment

FIG. 2 is a flowchart of meal planning and recipe recommendation system.

FIG. 3 is a flowchart of a multimedia content acquisition method.

FIG. 4 is a flowchart illustrating the components of the calorie and nutrition estimation system in a preferred embodiment.

FIG. 5 illustrates an example design to capture the plurality of images along with the side view and FIG. 5B along the top view using the sensors shown in FIG. 3.

FIG. 6 displays a plurality of images captured using a visible sensor.

FIG. 7 is a flowchart illustrating the object detection technique components using the classical approach.

FIG. 8 illustrates an example of a region of interest proposal technique.

FIG. 9 displays an example of a convolutional neural network.

FIG. 10 shows an example of the alpha trim convolution employed in the neural network. This removes the noise present in the images.

FIG. 11 shows an example of the classical convolution employed in the neural network. This retains the noise in the images.

FIG. 12 shows an example max-pooling layer employed in the neural network.

FIG. 13 shows an example response of the alpha-log-based activation function.

FIG. 14 shows an example response of the alpha-log positive-based activation function and its corresponding derivative function.

FIG. 15 is an example output of a model;

FIG. 16a shows an example of the steps involved during training an object detection neural network and FIG. 16b shows the steps involved during the testing phase of the neural network.

FIG. 17 shows an example of the anchor box generated for the object detection framework.

FIG. 18 shows an example flow chart of the 3-D reconstruction method.

FIG. 19 shows an example of super resolving a low-resolution image to obtain a high-resolution image using a neural network.

FIG. 20 is a flowchart of constructing a super-resolved image using deep learning techniques.

FIG. 21 shows an illustration of a possible network architecture of the super-resolution algorithm—(a) provides an example of the overall structure of super-resolution algorithm, (b) visualizes an example of the upsampling blocks used to perform ×2, ×3 and ×4 scaling, and (c) visualizes an example of the hypercomplex residual unit.

FIG. 22 is a flowchart for generating a segmentation mask using deep learning techniques.

FIG. 23 shows an illustration of a possible network architecture of the semantic segmentation algorithm—(a) provides an example of the overall structure of the network, (b) visualizes an example of the hypercomplex residual unit.

FIG. 24 shows an example of the segmentation masks a) is a set of the input visible images, (b) is the expected ground truth mask, (c) is the result obtained from traditional convolutional neural networks, and (d) is the result obtained from hypercomplex neural networks.

FIG. 25 shows an example of a 3-D point cloud obtained using images from FIG. 6.

FIGS. 26a and 26b show an example of the top and side view of the mesh generated from the 3-D point cloud with irrelevant objects.

FIGS. 27a and 27b show an example of the top and side view of the mesh after removing the irrelevant objects.

FIGS. 28a and 28b show an example of the top and side view of the mesh after the texture mapping process.

FIGS. 29a and 29b show an example of a food item's top and side view extracted from the 3-D model.

FIG. 30 is an example of the top view of the polygons generated for the 3-D model with color and mesh, and b visualizes the side view of just the mesh with the polygons.

FIG. 31 is a flowchart for generating a depth map using deep learning techniques.

FIG. 32 shows an example of (a) an original image and (b) the corresponding depth map generated using deep learning techniques.

FIG. 33 is an example of the tetrahedron, which is used to compute the volume of a 3-D model.

FIG. 34 is an example of a 2-D ellipse, and b is an example of a 2-D circle used for fitting the slices during volume estimation.

FIG. 35 is an example of volume estimation using the slicing method.

FIG. 36 is a flowchart of volume estimation using the deep learning method.

FIG. 37 is a flowchart of calorie estimation using the deep learning method.

FIG. 38 is an example flowchart of calorie estimation after training a deep learning network utilizing the ground truth calories provided by the nutrition experts.

FIG. 39 is an example of a new food database that can be used for calorie estimation along with other nutrients.

FIG. 40 is a schematic representation of an example computer that can perform at least a portion of the processing described herein.

### DETAILED DESCRIPTION

Embodiments of the present disclosure provide methods and systems for automated individual dietary planning incorporated with a dietary assessment, which may be customized based upon several unique characteristics specific to the dieter or group of dieters. Example architectures can be implemented on any of a wide array of commercially available computing devices, for example, smartphones, smartwatches, tablets, laptops, and/or desktops. These systems may or may not be connected to each other via any networks.

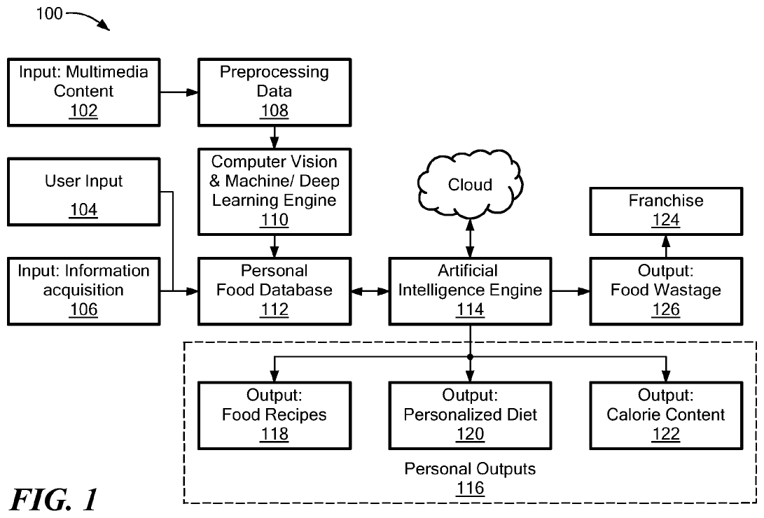

FIG. 1 illustrates a general block diagram 100 of example modules/steps for nutrition assessment and nutrition planning, according to an embodiment of the present disclosure. A general overview description of each component is now provided. Additional details of these components and their operation is provided below. The system may comprise a number of inputs that can be processed to generate nutrition information in accordance with example embodiments of the disclosure.

A first input 102 comprises multimedia content, which may comprise any form of visible, near-visible, thermal, grayscale, color, thermal imaging, and/or video information. A second input 104 may include user input which may comprise subjective opinions of a user/expert, for example, the user can set the weight-loss/gain goals, with a preference toward food items containing certain ingredients, accept/reject new recipes, accept/reject purchases of food items according to diet needs, etc. A third input 106 may include information acquisition which may comprise various objective information related to user's weight, body mass index (BMI), blood pressure, blood sugar level, basal metabolism that reflects muscle mass, measurement of motion quantity via user's movement linked to smartphone's GPS and gyro sensor, type and configuration of food consumed if the user is keeping a record of consumed food, each user's genetic information, a metabolic process which microorganisms and food react to, etc. A preprocessing module 108 and a computer vision and machine/deep learning engine 110 may comprise image object detection, thresholding, binarization, image segmentation, image multilevel binarization, image classification, image enhancement, image brightness, and darkness equalization, and/or image/video applications. A personal food database 112 may include database information on the menu, food, and ingredients, basic information for making food, calories consumed, nutrient intake, etc., food that can be made when ingredients are combined in the right order, and/or relevant recipes. An artificial intelligence engine 114 may comprise processing for finding the optimum menu per user by considering limiting conditions that reflect the status of each user based on the given inputs, algorithms that provide a suggestion about meal planning, algorithms for suggesting food items to buy, and/or food items that expire.

Embodiments of the system 100 can generate a range of outputs. In the illustrated embodiment, the system 100 outputs comprise personalized outputs 116 that can include, for example, food recipes 118, which may be dependent on the user inputs, a personalized diet 120 according to the goals, and/or calorie content 122 that may include an amount of calories consumed or nutrient intake. The personalized diet 120 and nutrient intake can be used by medical practitioners for diagnosis and can be used during treatment. In some embodiments, outputs can further include franchises 124 regarding the amount of food wasted 126 or consumed by the customer.

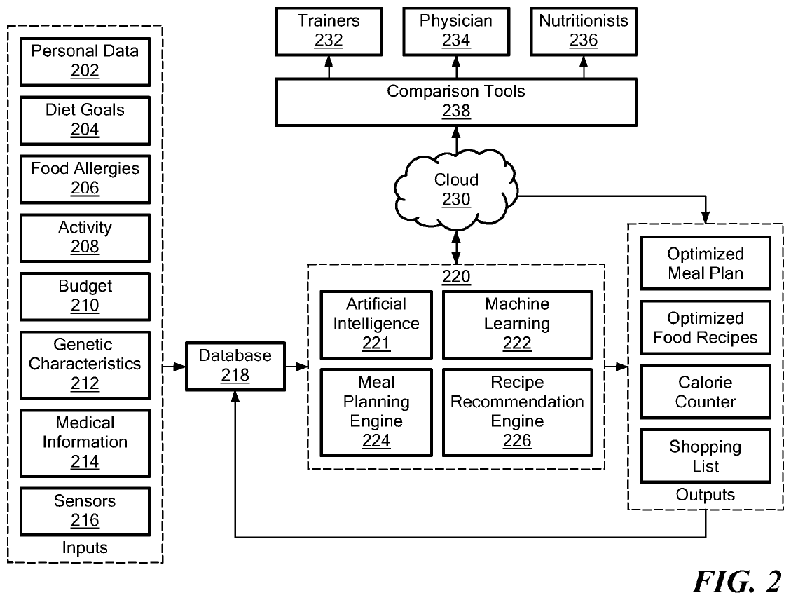

FIG. 2 shows an example automated individual dietary planning system 200 incorporated with a recipe suggestion engine. Inputs to the system can comprise a variety of types of information. Personal data 202 can include basic contact information such as name, address, telephone, and email. These aid in personalized database generation to synchronize with different practitioners (for example, trainers, doctors, nutritionists) accounts for diagnosis, and/or tracking purposes. Personal data 202 may also comprise birth date, gender, height, weight, desired weight, if pregnant or nursing, size measurements of biceps, triceps, forearms, neck, chest, waist, hips, upper thigh, mid-thigh, knee, calves, ankle, foot, dress size, shirt size, pant size, metabolic heart rate, current body fat percentage, desired body fat percentage, desired calories consumption, etc.

Diet goals 204 can comprise a user-provided number of servings, PFC (protein, fat, carb) ratios, and nutrients per person, Carb ratios (the daily carb ratio is the percentage of high complex carbohydrates versus low complex carbohydrates allowed on a diet), sugar, ingredient replacement suggestion, set alarms, set success criteria, desired date to reach the desired weight, indicate if the goal is to lose weight, gain weight, build muscle mass, or follow a strict eating plan for basic nutritional value or to treat a medical condition.

Food allergen data 206 can comprise user-provided individual food allergies, food they dislike, food intolerances, religious dietary requirements, cultural preferences, food preferences (for example, cooked well/med/done), etc.

Activity data 208 can comprise the current daily activity level, for example, jogging, running, swimming, hiking, yard yoga, paddleboarding, kayaking, etc., planned exercise goals, exercise specific to a muscle group category such as abs, back, chest, legs, shoulders, etc.

Budget data 210 can comprise a budget for groceries and dining, preparing a menu plan before shopping, considering special dietary needs such as dietary restrictions or gluten-free.

Genetic characteristics 212 can comprise user/physician-provided individual genotype that contributes to affecting appetite, satiety (the sense of fullness), metabolism, food cravings, body-fat distribution, and the tendency to use eating as a way to cope with stress. Medical information 214 can comprise user-provided physician name, phone, address, email, current medicines or vitamin supplements, medical conditions (current illnesses, diseases, and history of dieters and US 2006/O 1991 SS A1 family's medical conditions), blood type, chemistry, cholesterol, blood sugar, Berkley CHD profile, chorisol stress hormone, serotonin, TSHT3 & TSH T4 thyroid, Polycystic ovary syndrome, lepton, white blood cell count, red blood cell count, urine specific gravity, urine protein, urine ketones, urine glucose, uric acid, transferring saturation, protein/total serum, potassium, phosphorous, neutophils, monocytes, magnesium, lymphocytes, iron/serum, iron-binding capacity, hemoglobin, hemotocrit, glucose, globulin, eosinophilis, calcium/blood, basophiles, albumin, alcohol, smoking, menstruation, ovulation, blood pressure, body temperature, etc.

Sensors 216 data can comprise integrated information from different wearable health monitoring sensors to track Heart rate, number of steps, calories burned, blood pressure, etc.

The inputs may be integrated to generate a personal database 218 that may contain data records indicative of previously generated meal plans/recipes, user feedback on these meal plans/recipes, the information supplied by the user in connection with the generation of meal plans, and other data saved in connection, number, and type of meals per day, calories tracked over time, individual's preferences, crucial information such as dietary restrictions, allergens, etc. Personal data, along with information in the database, may be used to generate a generic meal plan by using the Recommended Dietary Allowance (RDA), Adequate Intake (Ai), Tolerable Upper Intake Level (UL) and Estimated Average Requirement (EAR) sources.

A processing module 220 may be configured to use one or more algorithms to find optimum results according to the user's conditions. In the illustrated embodiment, the processing module 220 includes an artificial intelligence (AI) module 221 and a machine learning algorithm (MLA) module 222 that may include genetic algorithms, decision trees, support vector machines, K-means clustering, etc., emergent technologies such as artificial neural networks that include feedforward neural network, multilayer perceptron, recurrent neural networks, etc. The MLA module 222 may be used to aid a meal planning engine (MPE) 224 and a recipe suggestion/recommendation engine (RRE) 226 to select an optimal meal/recipe. For example, the MPE 224 may provide meal suggestions to the user according to the preferences or suggest the user to have a particular food item at a restaurant setting that may complete the daily nutrition requirement. Once the meal planning is completed, the RRE 226 can find recipes that are best suited to the ingredients available or create a shopping list of the ingredients unavailable. Cloud services 230 may be used to obtain recipes along with nutrient content and serving sizes. The recommended recipes may be ordered from most to least similar to the user's preferences. The user may select one or more recommended recipes. These selected recipes are then divided into meals, food items, snacks, beverages along with their respective calorie content or nutrition amount per meal.

In some embodiments, recipes can be listed/suggested for breakfast, lunch, snacks, and dinner such that it meets the user-provided calories and nutrients and may be compared with the database with the RDA, Ai, UL, and EAR tables after every meal to check if the daily nutritional value is within the allowable range. If not, the MLA 222 is updated such that the next meal(/s)/recipe includes nutrients that were out of range in the previous meal. This process is repeated until the calorie, nutritional and protein, carbohydrate, fat ratio standards are met.

According to D'Adamo, Peter J., and Catherine Whitney. The genotype diet: Change Your Genetic Destiny to live the longest, fullest, and healthiest life possible. Harmony, 2007, genotypes can be broadly classified into an explorer, gatherer, hunter, nomad, teacher, or warrior. Each genotype has various requirements; for example, explorer needs to be limited to a few common food items such as ham, bacon, and most eggs and cheeses, Gatherers must limit most red meats and poultry, as well as many nuts, seeds, and legumes, etc. The MPE 224 may also keep in account the genotype of the individual and update the diet design appropriately. Furthermore, the MPE 224 considers the budget provided by the user, considers the activity levels, allergens, dietary restrictions, diet goals, and generates a shopping list with the quantity of food items required. The MPE 224 and RRE 226 may also consider the outputs of the sensors 216. For example, a blood sugar monitor can provide the user's current sugar level and insulin levels and make changes to the meal plans for recipes accordingly.

These generated meal plans and recipes provided to individuals or groups of individuals can be shared with professionals such as physicians 232, trainers 234, and nutritionists 236. A compare tool 238 can help these professionals to compare various meals, diets, ingredients in terms of cost, restrictions, allergies, etc. Furthermore, these professionals can access a patient's account to check their food habits for diagnosis purposes and recommend changes in the users' diet. The professionals 232, 234, 236 may also include the current treatments and diagnosis such that the MPE 224 and RRE 226 can provide natural remedies to aid the treatment process.

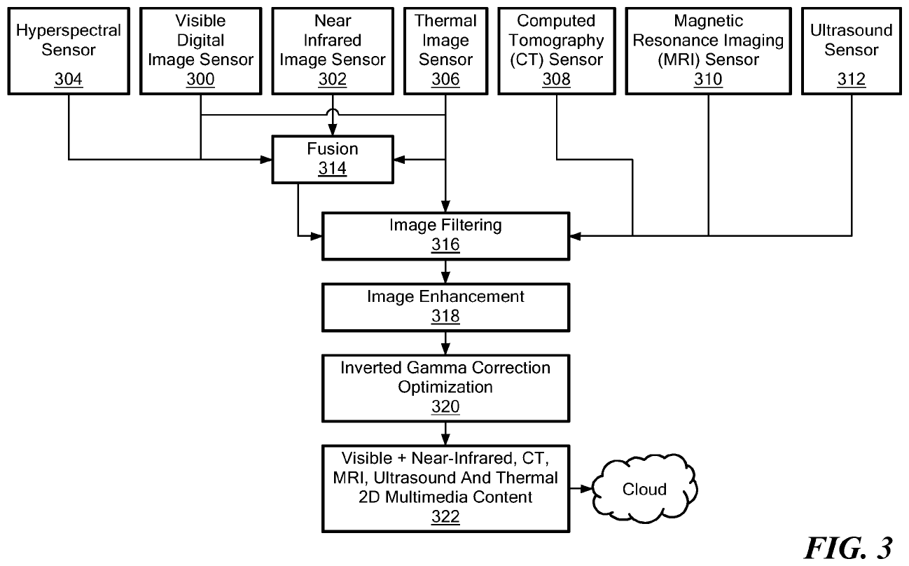

FIG. 3 shows example multimedia data acquisition according to some embodiments of the disclosure for two-dimensional (2D) and/or three-dimensional (3D) digital images from one or more sensors. Initial image data can be acquired via a 2D visible digital image sensor 300, a near-infrared image sensor 302, a hyperspectral sensor 304, a thermal image sensor 306, computed tomography sensor (CT) 308, magnetic resonance imaging (MRI) sensor 310 and/or ultrasound sensor 312. The visible, near-infrared, and thermal image data can be fused together by a fusion module 314, such as fusing either visible and thermal and/or visible and near-infrared. The resulting fused image, and/or images from other sensors, may contain different types of noise, for example, Gaussian noise, salt and pepper noise, speckle noise, anisotropic noise, etc. This noise can be filtered by an image filter 316 by applying one or more filters depending on the noise present, such as Gaussian filters to remove Gaussian noise, a median filter to remove salt and pepper noise, and/or other filters.

The filtered image can then be enhanced by an enhancement module 318, resulting in an output image or images, such as a visible and near-infrared, thermal, CT, MRI, and ultrasound 3D image and 2D image. The output images I can be corrected by a correction module 320 using optimized inverted gamma correction, for example. In example embodiments, this can be formulated, as shown in Equation 1.

$$f ( I ) = max ( I ) \times {( \frac{I}{max ( I )} )}^{\frac{1}{γ}} \qquad Equation 1$$

The parameter y in Equation 1 can be optimized, for example, by using various quality measures as described in Panetta, Karen, Arash Samani, and Sos Agaian. “Choosing the optimal spatial domain measure of enhancement for mammogram images.” Journal of Biomedical Imaging 2014 (2014): 3, Panetta, Karen, Eric Wharton, and Sos Agaian. “Parameterization of logarithmic image processing models.” IEEE Tran. Systems, Man, and Cybernetics, Part A: Systems and Humans (2007), which are incorporated herein by reference. The corrected output image(s) 322 form multimedia content, which may include visible, near-infrared, CT, MRI, ultrasound, and/or thermal 2D information, may then be stored in cloud storage or other types of memory. These stored output images can be used for display and/or with an image analytics system. For example, in some applications, the output images can be retrieved by an acquisition module and used as input images.

For the entirety of the present disclosure, operators which include but are not limited to +, −, ×, ÷ can be considered as classical operations (for example, arithmetic addition, subtraction, etc.), PLIP based operations, logarithmic based operations, and/or symmetric logarithmic based operations.

Alternatively, in some embodiments, image filtering, image enhancement, and inverted gamma correction optimizations steps can be skipped, and the filtered image may be stored in cloud storage, internal memory, or other types of memory. Furthermore, while the above image acquisition method is illustrated and described herein, it is within the scope of this disclosure to provide different types of image acquisition methods and methods configured to provide image data for use with one or more methods of the present disclosure. In other words, input images for use with the methods described herein are not limited to those acquired by the above-described system and method.

According to some embodiments, the present disclosure includes a set of building blocks for deep learning methodologies. Examples of building blocks may comprise, but are not limited to, convolutional layers, pooling layers, normalization layers, and fully connected layers (Goodfellow, Ian, et al. Deep learning. Vol. 1. Cambridge: MIT press, 2016).

Visual representation is provided in FIG. 9. The building blocks may be stacked together to form a network. These networks are trained via forward passes and backward-propagation, i.e., the computed gradients are added to the weighted neurons in the previous layer, which is further considered during training. The network is trained after the cost-function is minimized. Convolutional neural networks (CNN) may be used for deep learning with MC data. They are similar to neural networks and are made up of neurons that have learnable weight and biases. Each neuron receives an input, performs few operations, and is optionally followed by a non-linear operation. CNNs include an input and output layer and a multitude of hidden layers between the input and output. In each layer, activation volumes are altered with the use of differentiable functions. The building blocks described are applicable but not limited to any hypercomplex algebra, including but not limited to complex, tessarines, coquaternions, biquaternions, exterior algebras, group algebras, matrices, octonions, quaternions, and vector maps. For simplicity of exposition, consider the case of quaternions: the extension to different hypercomplex is straightforward.

Quaternion numbers refer to a four-dimensional generalization of the two-dimensional (2-D) complex algebra. The set of quaternion numbers is a part of the hyper-complex numbers, which are constructed by adding two more imaginary units in addition to the complex numbers. It consists of a scalar or real part q₀∈ [symbol] , a vector or imaginary part \vec{q}=(q₁, q₂, q₃)∈ [symbol] ³ and i, j, and k are the standard orthonormal basis for [symbol] ³. Then a quaternion [symbol] can be represented as shown in the equations below:

[symbol]

≡[q₀,\vec{v}],q₀∈ [symbol] ,\vec{v}∈ [symbol] ³ Equation 2

[symbol]

≡[q₀,q₁,q₂,q₃],q₀,q₁,q₂,q₃∈ [symbol] Equation 3

[symbol]

={q=q₀+q₁i+q₂j+q₃k|qₜ∈ [symbol] ,i²=j²=k²=ijk=−1} Equation 4

In this quaternion space, when q₀ is 0, [symbol] is a pure quaternion. One of the ways to construct a quaternion matrix is by utilizing a 4×4 orthogonal representation [A. M. Grigoryan and S. S. Agaian, Quaternion and Octonion Color Image Processing with MATLAB, p. 404, SPIE, vol. PM279, Apr. 5, 2018. [ISBN: 9781510611351] G. GüNAŞTI, “Quaternions Algebra, Their Applications in Rotations and Beyond Quaternions,” ed, 2012.]. This can be summarized into the following matrix of real numbers:

$$q ≡ [ \begin{matrix}{q}_{0} & - {q}_{1} & - {q}_{2} & - {q}_{3} \\ {q}_{1} & {q}_{0} & - {q}_{3} & {q}_{2} \\ {q}_{2} & {q}_{3} & {q}_{0} & - {q}_{1} \\ {q}_{3} & - {q}_{2} & {q}_{1} & {q}_{0}\end{matrix} ] \qquad Equation 5$$

The Hamilton product is the fundamental criterion in Quaternion CNNs to remodel vectors while maintaining the affine transformations such as translation, scaling, and rotation in the 3-D space [W. Hamilton, “On quaternions; or on a new system of imaginaries in algebra, letter to John T,” Graves (October 1843), 184]. The extension of quaternion multiplication to convolution is described further below.

Convolution operations are linear operations that can handle inputs of varying sizes. The input is usually a two-dimensional array of data, and the kernel may be a two-dimensional array of learnable parameters. In CNNs, the two-dimensional inputs and kernels are images. The convolution operation in the simplest case, the output value of the layer with input size (N, C_{ι}, H_{ι}, W_{ι}) and output (N, C_{ϕ}, H_{ϕ}, W_{ϕ}) can be precisely described as shown below

$$Y ( {N}_{i} , {C}_{{ϕ}_{j}} ) = ℬ ( {C}_{{ϕ}_{j}} ) + {\sum }_{λ = 0}^{{C}_{ι} - 1} W ( {C}_{{ϕ}_{j}} , λ ) ⊗ I ( {N}_{i} , λ ) \qquad Equation 6$$

where ⊗ is 2-D convolution, I is the multimedia content considered for convolution, N is batch size, C denotes the number of channels, H is a height of input planes in pixels, and W is the width in pixels, [symbol] is the bias, [symbol] is the weights.

As seen in Equation 6, convolution operations in a real-valued domain are performed by convolving a vector with a randomized weight. Similarly, in quaternion space, convolution can be achieved by applying quaternion weights on a quaternion vector. However, this is not straightforward and requires manipulation of real-valued matrices. Let [symbol] = [symbol] + [symbol] i+ [symbol] j+ [symbol] k be a quaternion input, and [symbol] = [symbol] + [symbol] i+ [symbol] j+ [symbol] k a quaternion weight, the quaternion convolution can be defined as

[symbol]

⊗ [symbol] ≡ [symbol] −[ [symbol] + [symbol] + [symbol] ]+ [symbol] + [symbol] + [symbol] − [symbol] ]i+[ [symbol] − [symbol] + [symbol] + [symbol] ]j+[ [symbol] + [symbol] − [symbol] + [symbol] ]k Equation 7

This can also be represented in a matrix form by incorporating Equation 5:

$$𝕎 ⊗ 𝒥 ≡ [ \begin{matrix}𝕣 & - 𝕩 & - y & - 𝕫 \\ 𝕩 & 𝕣 & - 𝕫 & 𝕪 \\ 𝕪 & 𝕫 & 𝕣 & - 𝕩 \\ 𝕫 & - 𝕪 & 𝕩 & 𝕣\end{matrix} ] ⊗ [ \begin{matrix}ℛ \\ 𝒳 \\ 𝒴 \\ 𝒵\end{matrix} ] = [ \begin{matrix}ℛ′ \\ {𝒳}^{′} i \\ {𝒴}^{′} i \\ {𝒵}^{′} k\end{matrix} ] \qquad Equation 8$$

Note that the output of the quaternion is produced by convolving each unique linear combination of the weight ( [symbol] ) with each axis of the input. This is due to the structure of quaternion multiplication, which enforces cross interactions between each axis of the weight and input. It can be noted that quaternion convolution can be performed by utilizing standard convolution and depth wise separable convolution.

Alternatively, according to some embodiments, the present disclosure includes systems and methods for alpha-trimmed based convolution. This convolution layer can be defined as shown in Equation 9

$$y ( {N}_{i} , {C}_{{ϕ}_{j}} ) = ℬ ( {C}_{{ϕ}_{j}} ) + {\sum }_{λ = 0}^{{C}_{ι} - 1} {f}_{α} ( W ( {C}_{{ϕ}_{j}} , λ ) ) ⊗ {f}_{α} ( I ( {N}_{i} , λ ) ) \qquad Equation 9$$

$${f}_{α} ( x ) = \{ \begin{matrix}{x}_{sort} , & α \leq {x}_{sort} \leq n - α ; n > α \\ ψ , & otherwise\end{matrix} \qquad Equation 10$$

$${f}_{α} ( x ) = \{ \begin{matrix}ψ , & α \leq {x}_{sort} \leq n - α ; n > α \\ {x}_{sort} , & otherwise\end{matrix} \qquad Equation 11$$

where ⊗ is 2-D convolution, I is the multimedia content considered for convolution, N is the batch size, C denotes the number of channels, H is a height of input planes in pixels, and W is the width in pixels, [symbol] is the bias, [symbol] is the weights, ƒ_{α}(x) is the alpha-trimming function, n is the maximum length of x. ψ can be replaced with either zero, constant, or replication of the nearest value.

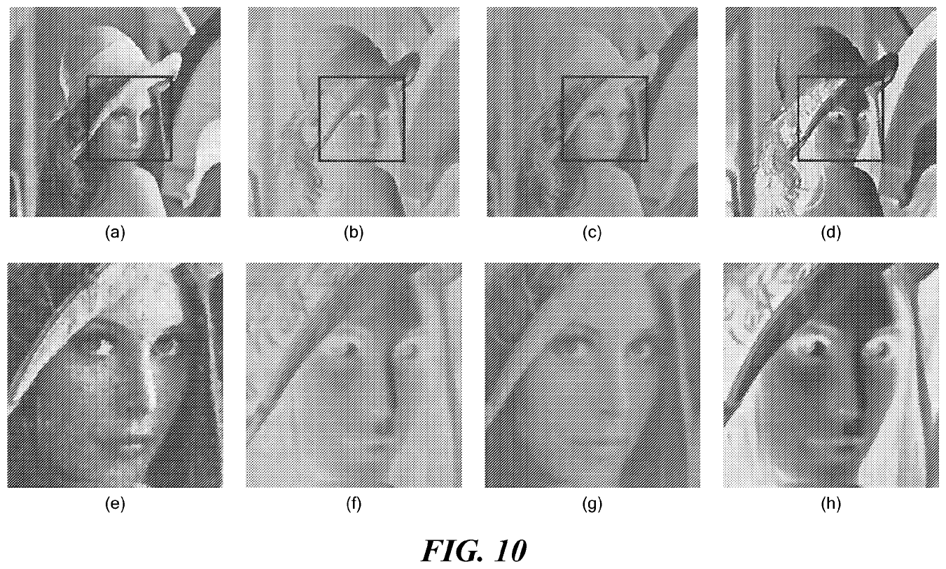

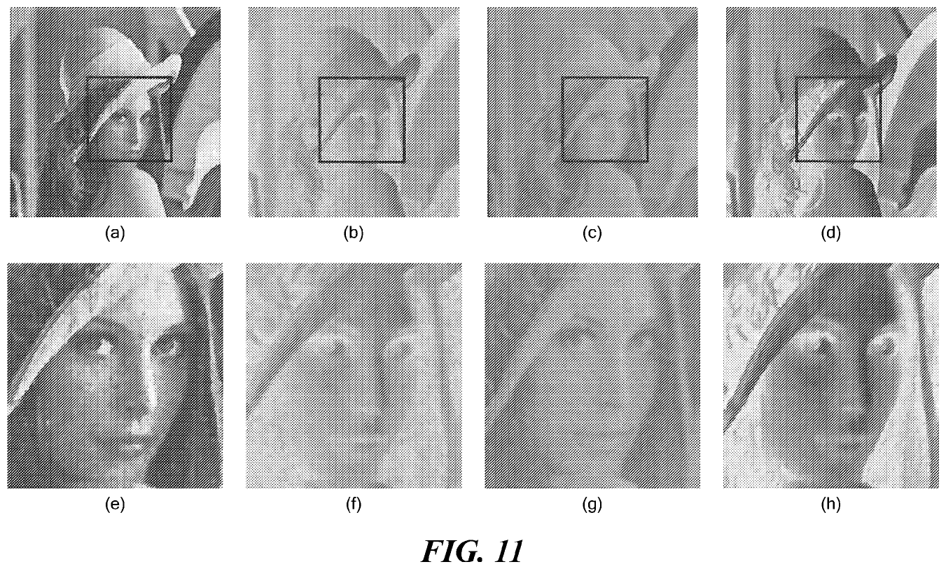

The alpha trimming function ƒ_{α}(x) can be replaced with inner trimmed function (Equation 10) or outer trimmed function (Equation 11). In some cases, inner trimmed function can be used for alpha trimming weights, and outer trimmed function can be used for alpha trimming inputs or vice versa. The alpha-trimmed based convolution has advantages that include, but are not limited to, restoration of signals and images corrupted by additive non-Gaussian noise. They can be employed in circumstances where the input has a noise that deviates from Gaussian with impulsive noise components. A visual comparison between alpha trimmed convolution and classical convolution can be seen in FIG. 10, and FIG. 11, respectively. FIG. 10a, and FIG. 11a is the noisy input to the convolution layer and FIG. 10e, and FIG. 11e is the zoomed view of the black box from FIG. 10a, and FIG. 11a respectively. FIGS. 10b-d and FIGS. 11b-d are the output of alpha trimmed convolution and classical convolution, respectively, while FIGS. 10f-h and FIGS. 11 f-h is the corresponding zoomed view. It can be seen in FIG. 10 that the alpha trimmed convolution has reduced most of the noise from the input while the classical convolution was unable to eliminate noise. From here on, convolution can comprise a traditional CNN, hypercomplex CNNs, alpha trimmed CNN, and/or a combination of these and is denoted as T/A/H convolutional layer.

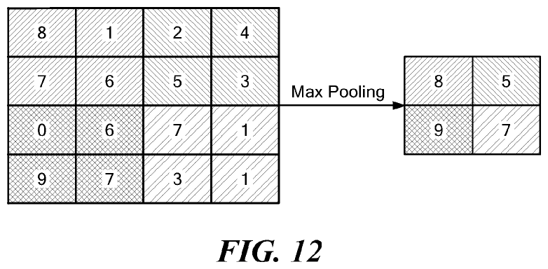

Pooling layers summarize the neighborhoods of output units and replace them with one value in the kernel map. Its function is to progressively reduce the spatial size of the representation to reduce the number of parameters and computation in the network. This improves results due to less overfitting. The pooling layer operates independently on every depth slice of the input and resizes it spatially. The most common form is a pooling layer with filters of size 2×2 applied with a stride of 2 downsamples every depth slice in the input by two along both width and height, discarding 75% of the activations. The most widely used pooling operations are ‘max-pooling’ and ‘average pooling.’ An example of max pooling applied to a matrix can be seen in FIG. 12. These can be used for traditional or alpha-trimmed neural networks. In the case of hypercomplex space, the average pooling of the real part and the imaginary parts of the hypercomplex matrix separately will not affect the pooling result. However, in terms of max or min-pooling, pooling each piece individually will create a data mess. Because the position of the maximum or minimum for each axis, i.e., r, i, j, and k, (in the case of a quaternion) is different, algorithms to get the guidance matrix for the hypercomplex matrix is required. The guidance matrix can be computed using:

guidance=Θ(q₀^{ψ}+q₁^{ψ}+q₂+ [symbol] Equation 12

where Θ can include any mathematical operation such as maximum, minimum, absolute, ψ and ω̅ are parameters utilized to aid in constructing the guidance matrix.

The Normalization layer is used to normalize the input layer by adjusting and scaling the activations. For example, when a few features range from 0 to 1 and some from 1 to 1000, normalize them helps to speed up learning. The most widely used normalization layer is batch normalization. [Ioffe, Sergey, and Christian Szegedy. “Batch normalization: Accelerating deep network training by reducing internal covariate shift.” arXiv preprint arXiv:1502.03167 (2015).]. This technique normalizes the output of a previous activation layer by subtracting the batch mean and dividing by the batch standard deviation.

Alternatively, any normalization technique such as group normalization, switchable normalization, layer normalization, instance normalization techniques that can learn different normalization operations for different normalization layers can be employed. A few of the aforementioned normalization techniques, such as batch normalization, group normalization techniques, cannot be extended without modifications for hypercomplex space. For example, in the case of a quaternion batch normalization, the mean needs to be computed along each r, i, j, and k axis. In contrast, the variance needs to be computed across all the axis. One of the ways to compute the quaternion batch norm is proposed by Gaudet, Chase J., and Anthony S. Maida. “Deep quaternion networks.” 2018 International Joint Conference on Neural Networks (IJCNN). IEEE, 2018. Alternatively, an approach proposed by Wang, Jinwei, et al. “Identifying computer-generated images based on quaternion central moments in the color quaternion wavelet domain.” IEEE transactions on circuits and systems for video technology 29.9 (2018): 2775-2785. can also be employed.

According to some embodiments, the present disclosure includes hypercomplex-based weight normalization and standardization techniques. Alternately, a novel hypercomplex-based weight normalization technique can be applied. For explanation purposes, a quaternion hypercomplex is used. Consider a standard neural network where the computation of each neuron consists of taking a weighted sum of input features, followed by an elementwise nonlinearity:

y=Ø( [symbol] · [symbol] +b) Equation 13

where Ø(.) denotes an elementwise nonlinearity activation function, [symbol] is an n-dimensional quaternion weight, b is the bias and [symbol] is an n-dimensional quaternion input, and y is an n-dimensional quaternion output

To speed up the convergence of the hypercomplex neural network, reparameterization of each weight vector [symbol] in terms of a parameter vector V and a scalar parameter G is defined. The weight vectors can be expressed in terms of the new parameters using Equation 14.

$${𝕎}_{x} = \frac{G}{‖ V ‖} {V}_{x} ∀ x = r , i , j , k \qquad Equation 14$$

where V is a k-dimensional vector, G is a scalar, and ∥V∥ denotes the quaternion norm. This reparameterization has the effect of fixing the quaternion norm of the weight vector ∥V∥ and thus we have ∥ [symbol] ∥=G, independent of the parameters V. (A. Greenblatt, S. Agaian, Introducing quaternion multi-valued neural networks with numerical examples, Information Sciences Volume 423, January 2018, Pages 326-342)

This weight normalization technique improves the conditioning of the gradient and leads to improved convergence of the optimization procedure: Better speed of convergence is achieved by decoupling the quaternion norm of the weight vector (G) from the direction of the weight vector (V/∥V∥).

Weight standardization is a technique that considers the smoothing effects of weights more than just length-direction decoupling. It aims at reducing the Lipschitz constants of the loss and the gradients. The main difference between traditional weight standardization and quaternion weight standardization is the way, mean and standard deviation is computed. More formally, consider a quaternion convolutional layer as defined in Equation 7 or Equation 8, then the weight standardization can be defined as shown in Equation 15.

$$= WS ( 𝕎 ) \qquad Equation 15$$

$$where WS ( {𝕎}_{p} ) = \frac{{𝕎}_{p} - {μ}_{p}}{σ} ∀ p = r , i , j , k \qquad Equation 16$$

$$\begin{matrix}μ = \frac{1}{N} {\sum }_{1}^{N} {q}_{0} + {q}_{1} i + {q}_{2} j + {q}_{3} k \\ = {\overline{q}}_{0} + {\overline{q}}_{1} i + {\overline{q}}_{2} j + {\overline{q}}_{3} k\end{matrix} \qquad Equation 17$$

$$σ = \sqrt{\frac{1}{N} {\sum }_{1}^{N} Δ {q}_{0}^{2} + Δ {q}_{1}^{2} + Δ {q}_{2}^{2} + Δ {q}_{3}^{2}} ; Δ {q}_{p} = {q}_{p} - {\overline{q}}_{p} \qquad Equation 18$$

A fully connected layer is similar to regular neural networks wherein neurons are fully connected to all activations in the previous layer. Their activations can hence be computed with a matrix multiplication followed by a bias offset. In the case of hyper-complex, quaternion being taken as an example, all parameters are quaternions, including inputs, outputs, weights, and biases.

Another major component of neural networks in activation functions. It is a non-linear transformation, i.e., it performs a transformation on the input such that the output values are within a manageable range. Generally, these functions are nonlinear and continuously differentiable. Nonlinearity allows the neural network to be a universal approximation; a continuously differentiable function is necessary for gradient-based optimization methods, which is what allows the efficient backpropagation of errors throughout the network.

Alternatively, according to some embodiments, the present disclosure includes systems and methods for α log activation layer. This can be formulated, as shown in Equation 19.

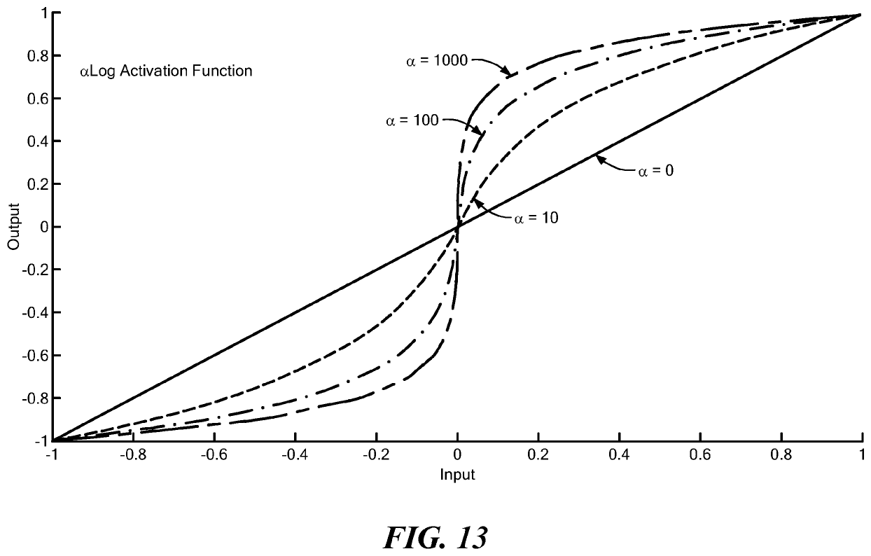

The characteristic curve can be visualized in FIG. 13.

$$f ( x ) = Δ \{ \begin{matrix}\frac{ln ( 1 + \ddot{α} x )}{ln ( 1 + \ddot{α} )} , & x > 0 \\ x , & x = 0 \\ \frac{ln ( 1 - \ddot{α} x )}{ln ( 1 + \ddot{α} )} , & x < 0\end{matrix} \qquad Equation 19$$

It can be seen from FIG. 13, that when α is 0, it acts like a linear function, and as α increases, higher the non-linearity. Alternatively, α log P activation layer (P stands for positive) can be defined as shown in. Its derivative is formulated in.

$$f ( x ) = Δ \{ \begin{matrix}\frac{ln ( 1 + α x )}{ln ( 1 + α )} , & x \geq 0 \\ ξ , & x < 0\end{matrix} \qquad Equation 20$$

$${f}^{′} ( x ) = Δ \{ \begin{matrix}\frac{α}{ln ( 1 + α )} \frac{1}{1 + α x} , & x \geq 0 \\ \frac{d ξ}{d x} , & x < 0\end{matrix} \qquad Equation 21$$

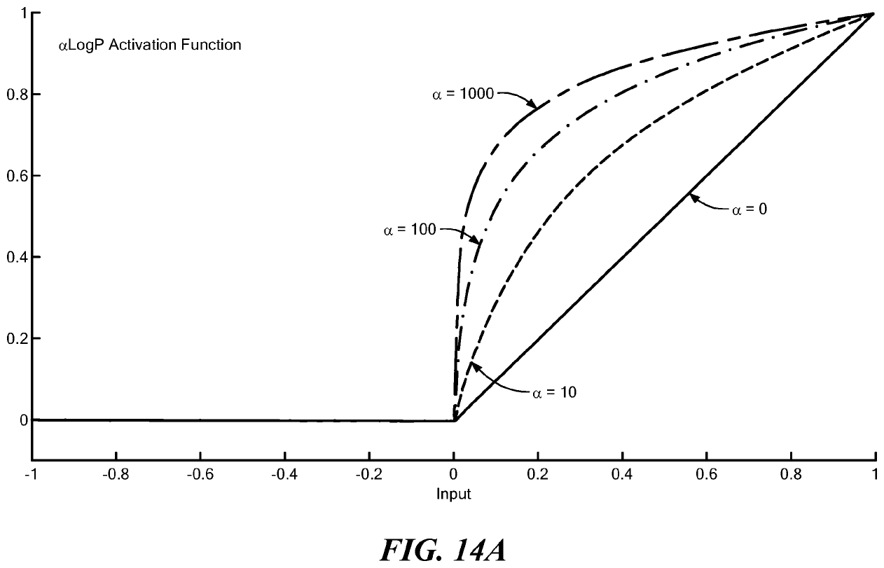

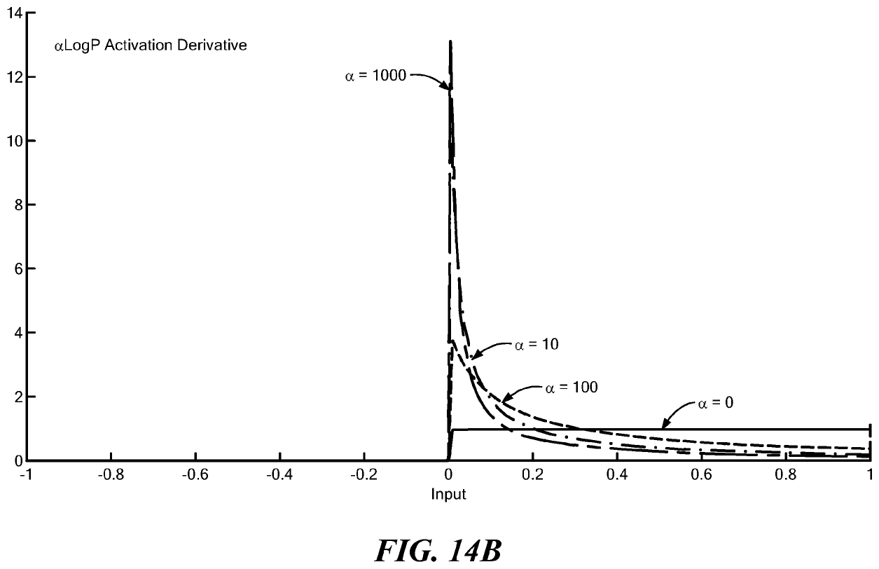

This activation function and its derivative function can be visualized FIG. 14a and FIG. 14b. When α=0, it is linear function with a max range of 1. When α<0, output is zero thus it has sparsity. As it ranges between 0 and 1, it does not cause blowing up as seen in ReLU activation function. Another approach is to use combine various activation functions for different intervals. It can be formulated, as shown in.

$$f ( x ) = Δ \{ \begin{matrix}Θ , & x \geq {t}_{h} \\ Λ , & {t}_{l} < x < {t}_{h} \\ K , & x \leq {t}_{l}\end{matrix} f ( x ) = Δ \{ \begin{matrix}Θ , & x \geq {t}_{h} \\ Λ , & {t}_{l} < x < {t}_{h} \\ K , & x \leq {t}_{l}\end{matrix} \qquad Equation 22$$

In Equation 22, Θ, Λ, K can be

$$\frac{ln ( 1 + \ddot{α} x )}{ln ( 1 + \ddot{α} )} , - \frac{ln ( 1 - \ddot{α} x )}{ln ( 1 + \ddot{α} )} ,$$

x or any equation that includes but not limited to other state-of-the-art activation functions such as sigmoid, tanh, ReLU, Leaky ReLU, etc.

Loss is an essential component of deep learning architectures. These are mathematical functions used to evaluate the fitting of the models. Higher loss generally implies poor model fitting. Using these losses, the weights/kernels and bias of the convolutional layers are updated.

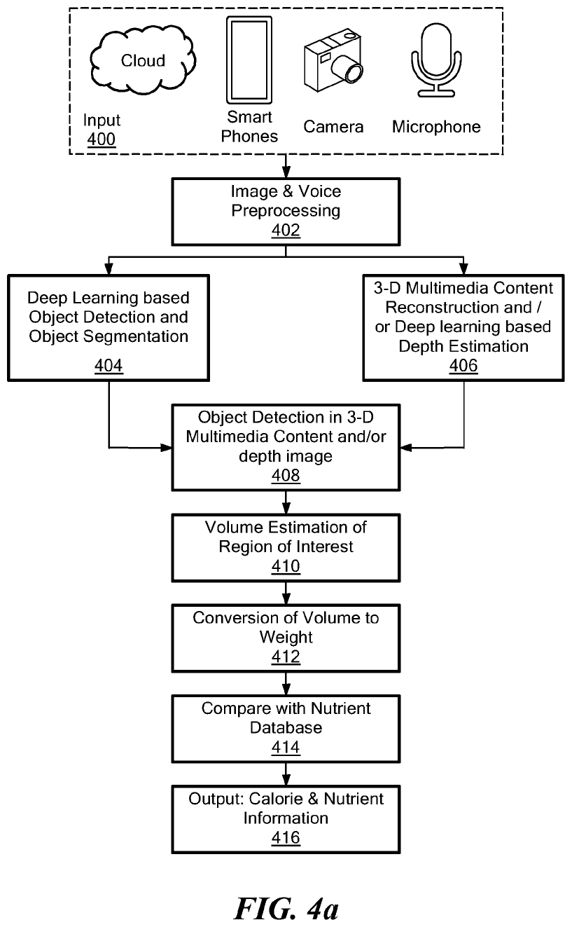

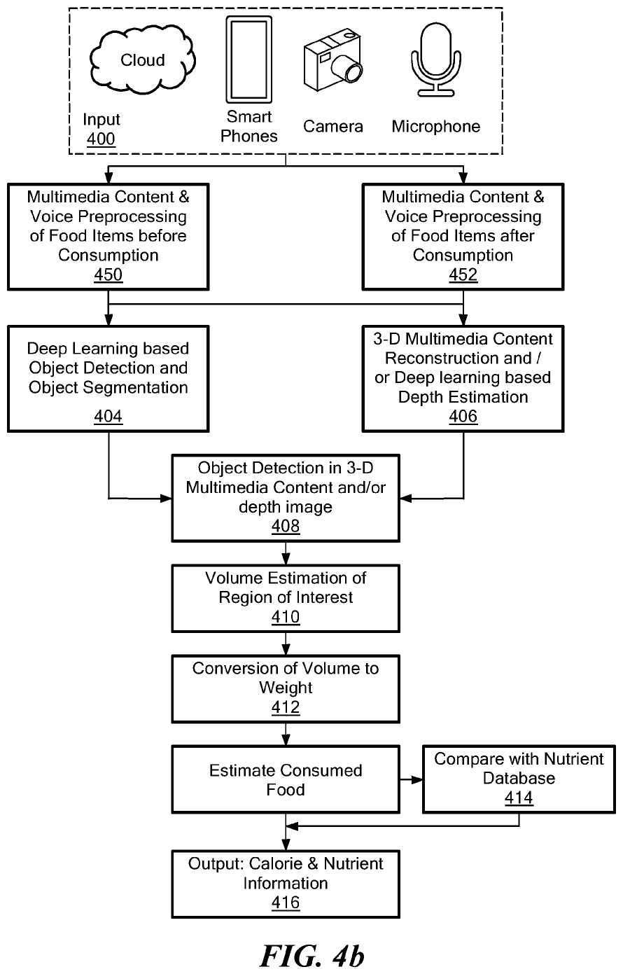

According to some embodiments, the present disclosure includes systems and nutrient and calorie estimation methods, as illustrated in FIG. 4. The multimedia content (MC) may be captured from any sensor mentioned in FIG. 3. Any device that contains one or more of these sensors along with a computing platform can be used, for example, smartphones, laptops, digital cameras, desktop computers or servers, etc. The MC may also be received through the cloud system. The MC captured may contain one or more images of the food plate and/or food items. The MC can be taken from any position without any specific angles. Additionally, the MC can be from different sources with a particular sensor type or various sources with varying kinds of sensors. For example, MC can be captured using a visible sensor from smartphones, digital cameras, and laptop, and can be combined for further processing. The MC can be captured using visible and thermal sensors in another example.

FIG. 4a shows example processing steps for nutrient and calorie estimation in accordance with illustrative embodiments of the disclosure. Input 400 from the cloud, smart phone, camera, microphone, or the like is received for image and voice preprocessing 402. In parallel, in step 404 deep learning base object detection and object segmentation is performed and in step 406, 3D multimedia content reconstruction and/or deep learning based depth estimation is performed. The outputs from the steps of 404 and 406 are processed in step 408 by performing object detection in 3D multimedia content and/or depth imaging. In step 410, volume estimation is performed for a region of interest and in step 412 the estimated volume is converted to weight. In step 414, the data is compared with information in a nutrient database. In step 416, calorie and nutrient information is output.

FIG. 4b is similar to the processing shown and described in FIG. 4b in which like reference numbers indicate like processing. In FIG. 4b, received input is processed in step 450 for multimedia content and voice preprocessing of food items before consumption. In step 452, received input is processed for multimedia content and voice preprocessing of food items after consumption. As described above, the before and after consumption data is processed in steps 408 and 406.

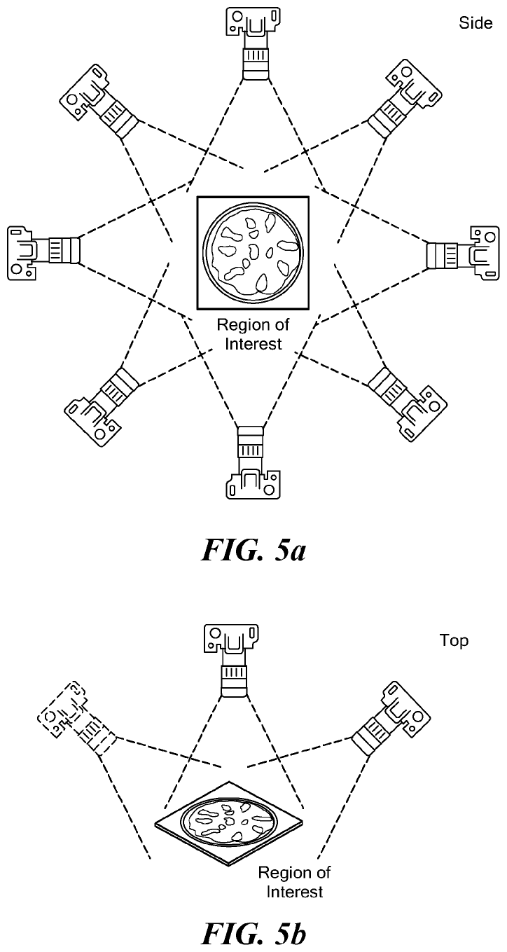

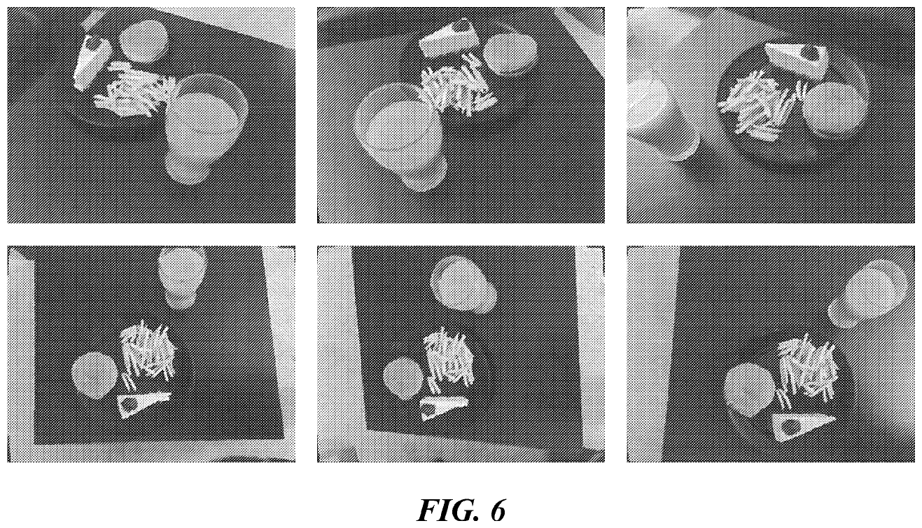

An example to obtain a plurality of images along the side views is illustrated in FIG. 5a and along the top view, is illustrated in FIG. 5b. An example of visible images taken from different positions is shown in FIG. 6. The MC may also be captured before consumption and after consumption of the food items. The MC may also be captured at multiple intervals of consuming food. The MC may be subject to parallax and varying illumination. The next step is to apply preprocessing techniques, including filtering, recoloring, color space conversion, image super-resolution, etc.

In some cases, the resulting image (and/or other image data if not initially fused), may contain different types of noise, for example, Gaussian noise, salt and pepper noise, speckle noise, anisotropic noise, etc. This noise can be filtered by applying one or more filters depending on the noise present, such as Gaussian filters to remove Gaussian noise, a median filter to remove salt and pepper noise, and/or other filters.

If the MC is grayscale, no color space transformation is necessary. However, if the input image is a color image, a suitable color space transformation can be applied. Specific color transformation models may be used for different color models such as CIE, RGB, YUV, HSL/HSV, and CMYK. Additionally, a color space model, median-based PCA conversion as described in Qazi, Sadaf, Karen Panetta, and Sos Agaian. “Detection and comparison of color edges via median-based PCA.” Systems, Man, and Cybernetics, 2008. SMC 2008. IEEE International Conference on. IEEE, 2008, may also be employed. Alternatively, —α-trim based principal component analysis, as described in Karen Panetta, Shreyas Kamath K. M, and Sos Agaian “Bio-Inspired multimedia analytic systems and methods” can be applied.

When the images are used as an input to hypercomplex networks, further preprocessing may be required. For example, in the case of a quaternion network, the input to the network requires four input channels, i.e., r, i, j, and k. As the inputs are generally visible images, it includes three channels. The fourth channel may include a grayscale image. In some cases, the inputs may comprise just one channel; in these cases, decomposition techniques such but not limited to ensemble empirical mode decomposition can be applied to generate a set of four channels. Or in the case of an octonion network, each channel in the color image can be decomposed into two channels, and eight channels can be generated. In a few other cases where multiple sensors exist, then the channels can be stacked together. For example, when a thermal image and visible image is available, then the thermal image can be considered as r axis, and the color image can be considered as i, j, and k axis. In a few cases when NIR and visible images are available, the grayscale of NIR with NIR image (total four channels) and the grayscale of visible with a visible image (total four channels) can be stacked together. It can be processed using an octonion convolutional network. In some cases where a visible image with depth image is available, these can be stacked together as input to a quaternion network. In a few other cases, each channel can be decomposed into wavelet transforms to provide a different set of images. For example, when a quaternion is used, 4 level wavelet decomposition can be performed, when octonion is used, 8 level wavelet decomposition can be performed. Furthermore, advanced nonlinear decomposition techniques such as EEMD as defined in [Bakhtiari, S., Agaian, S., & Jamshidi, M. (2011, April). A novel empirical mode decomposition-based system for medical image enhancement. In 2011 IEEE International Systems Conference (pp. 145-148). IEEE.] can also be employed to decomposed a given MC into different components and fed as input to the neural network.

According to some embodiments, the present disclosure includes super-resolution using hypercomplex neural networks. Image super-resolution is the task of inferring a high-resolution image with finer details from a low-resolution image. This recovers missing frequency details and removes the degradation that arises during the image capturing process. Furthermore, it extrapolates the high-frequency components and minimizes aliasing and blurring. This method can include but is not limited to deep learning-based super-resolution methods and non-deep learning based super-resolution methods. these methods take low-resolution images as input and provide a high-resolution image as output.

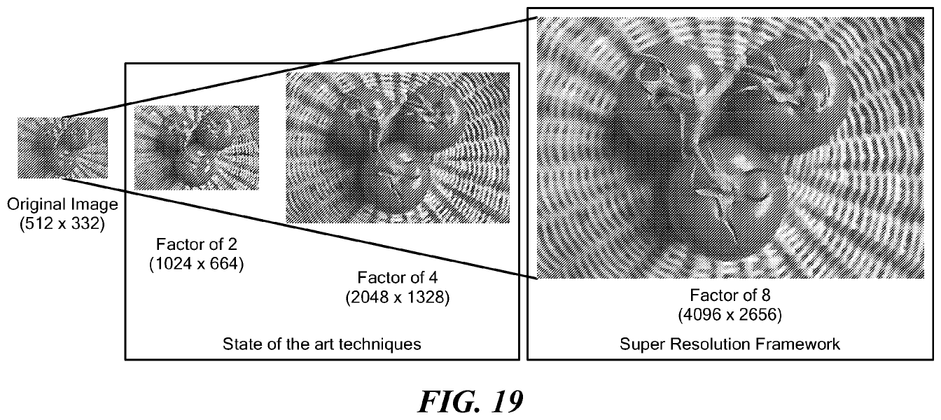

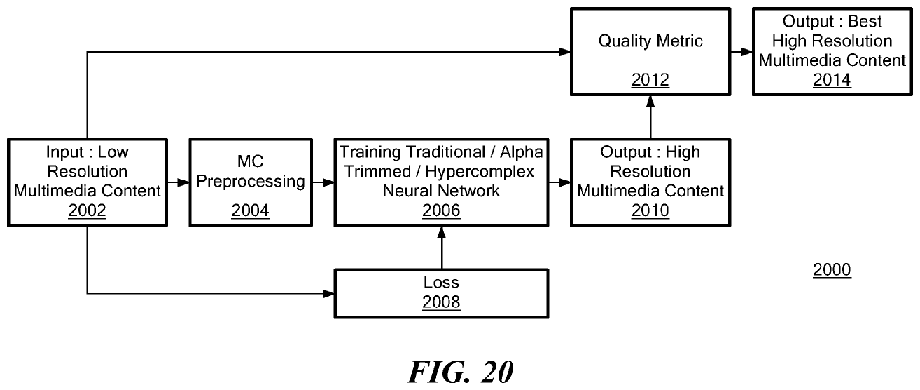

FIG. 19 shows an example of super resolving a low-resolution image to obtain a high-resolution image using a neural network. FIG. 20 shows an example flowchart 2000 for training deep learning-based super-resolution network. The low-resolution images 2002 are generally downsized versions of the ground truth. Furthermore, in some cases, as a part of preprocessing 2004, Gaussian kernel is applied to blur the image, or other kernels can be utilized to induce degradation. Neural network training 2006 can receive information from a loss function 2008 to generate an output in the form of high-resolution MC 2010, which can be provided to a quality metric 2012 for generation of best HR MC 2014.

The input low-resolution image I_{LR} of any arbitrary size (m,n) which has undergone degradation process (preprocessing) from its corresponding high-resolution image I_{HR} can be formulated as

I_{LR}= [symbol] (I_{HR};ψ) Equation 23

where ψ is a set of parameters utilized for the degradation process, which include scaling factor, noise intensity, blurring, and defocusing. In the case of deep learning-based super-resolution technique, the aforementioned T/A/H convolutional and other layers can be utilized. As activation layers, the aforementioned layers can be applied, or state-of -the-art techniques such as ReLU, LReLU can also be employed. The CNN structure can be of any fashion; for example, the CNNs can be structured serially or parallel. The loss function 2008 may include L1, mean squared error, structural similarity index (SSIM), multi-scale SSIM, the method in Nercessian, Shahan, Sos S. Agaian, and Karen A. Panetta. “An image similarity measure using enhanced human visual system characteristics.” Mobile Multimedia/Image Processing, Security, and Applications 2011. Vol. 8063. International Society for Optics and Photonics, 2011, Panetta, Karen, Arash Samani, and Sos Agaian. “Choosing the optimal spatial domain measure of enhancement for mammogram images.” Journal of Biomedical Imaging 2014 (2014): 3, Panetta, Karen, Eric Wharton, and Sos Agaian. “Parameterization of logarithmic image processing models.” IEEE Tran. Systems, Man, and Cybernetics, Part A: Systems and Humans (2007). Alternatively, methods such as Dong, Chao, Chen Change Loy, and Xiaoou Tang. “Accelerating the super-resolution convolutional neural network.” European Conference on Computer Vision. Springer, Cham, 2016, Lai, Wei-Sheng, et al. “Deep Laplacian pyramid networks for fast and accurate superresolution.” IEEE Conference on Computer Vision and Pattern Recognition. Vol. 2. No. 3. 2017, Chang, Hong, and Yeung, Dit-Yan and Xiong, Yimin, Super-resolution through neighbor embedding, CVPR, 2004, Freeman, William T, and Jones, Thouis R and Pasztor, Egon C, Example-based super-resolution, IEEE Computer graphics and Applications, 2002. Yang, Jianchao and Wright, John and Huang, Thomas S and Ma, Yi, Image super-resolution via sparse representation, IEEE trans. image-processing 2010. can also be employed. As an example, the hypercomplex based quaternion layer is utilized for description purposes.

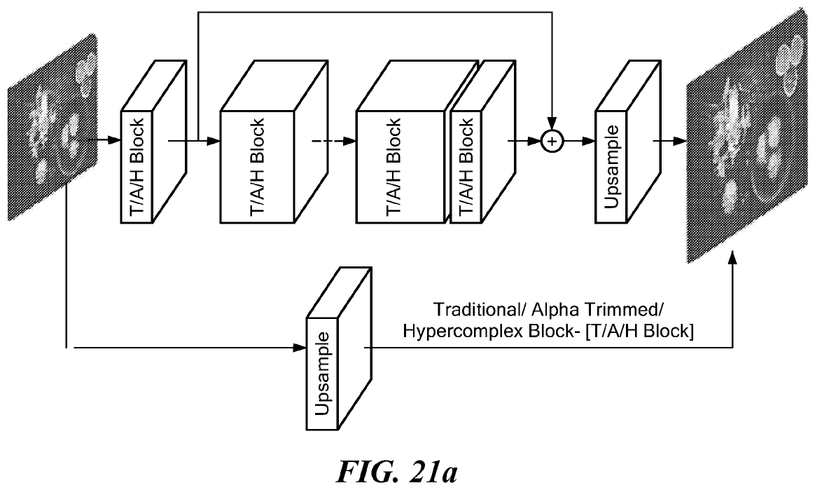

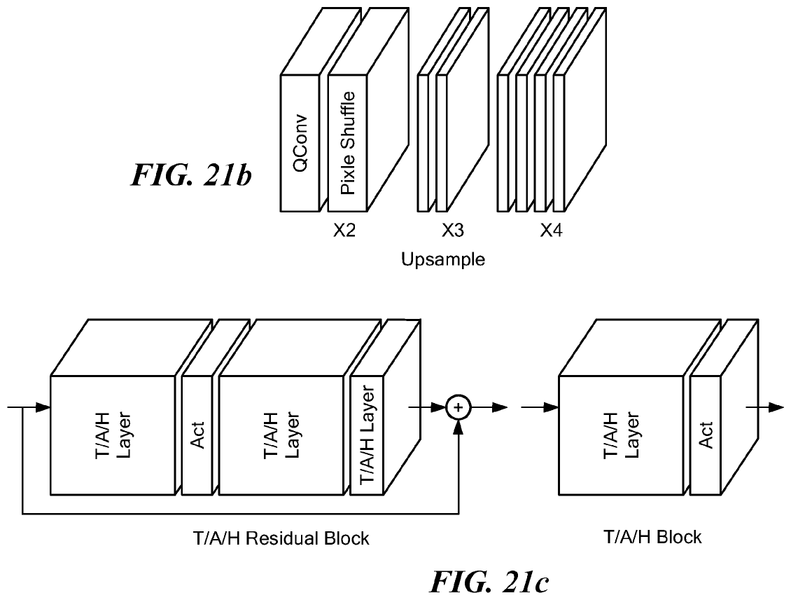

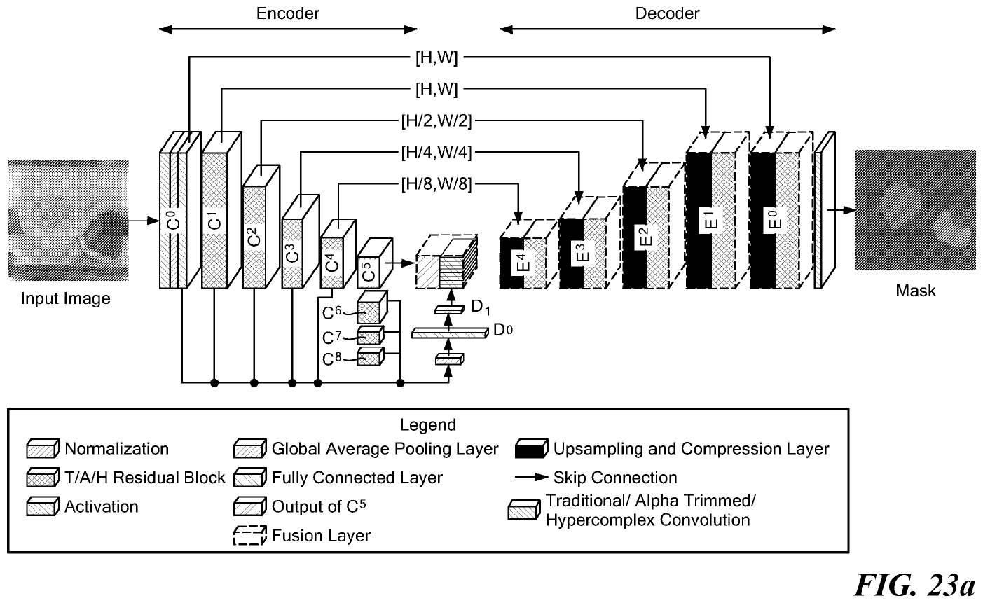

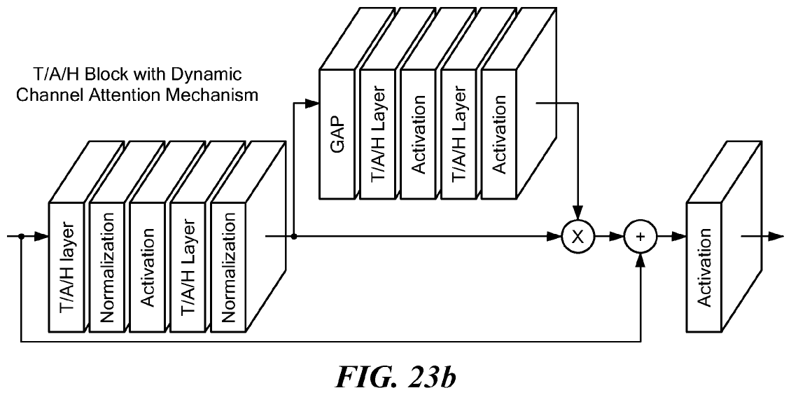

FIG. 21 (a) shows an example network that can be used for super-resolution and FIG. 21b shows an example upsample block. The input QI_{LR} is a degraded quaternion image which is used to obtain its high-resolution image QI_{HR}. Each T/A/H block can include a combination of T/A/H convolutional layers along with activation layers or a residual unit (QRU—quaternion as an example) displayed in FIG. 21c or with a dynamic channel attention mechanism, as shown in FIG. 23 (b). This block may be replicated N number of times serially or can also be designed in a nested fashion. Generally, the first convolutional layer extracts a set of quaternion feature space from the quaternion image space; the following QRUs can have channel dimensions as per the additive configuration, which can be formulated as

$${D}_{k} = \{ \begin{matrix}〈 η 〉 & if k == 1 \\ 〈 {Γ}_{k} 〉 & if 2 \leq k \leq N + 1\end{matrix} \qquad Equation 24$$

$$where {Γ}_{k} = \{ \begin{matrix}[ {D}_{k - 1} + 〈 \frac{α}{N} 〉 ] & {ρ}_{1} \\ [ {D}_{k - 1} \times β + 〈 \frac{α}{N} 〉 ] \vert 0.1 \leq β \leq 1 & {ρ}_{2} \\ η & {ρ}_{3}\end{matrix}$$

$$where {Γ}_{k} = \{ \begin{matrix}[ {D}_{k - 1} + 〈 \frac{α}{N} 〉 ] & {ρ}_{1} \\ η & {ρ}_{2}\end{matrix} if ρ < 2$$

Alternately, multiplication-based channel dimension configuration can be represented as:

$${D}_{k} = \{ \begin{matrix}〈 η 〉 & if k == 1 \\ 〈 {Γ}_{k} 〉 & if 2 \leq k \leq N + 1\end{matrix} \qquad Equation 25$$

$$where {Γ}_{k} = \{ \begin{matrix}[ {D}_{k - 1} + 〈 {α}^{\frac{1}{N}} 〉 ] & {ρ}_{1} \\ [ {D}_{k - 1} \times β + 〈 {α}^{\frac{1}{N}} 〉 ] \vert 0.1 \leq β \leq 1 & {ρ}_{2} \\ η & {ρ}_{3}\end{matrix} if ρ > 2$$

$$where {Γ}_{k} = \{ \begin{matrix}[ {D}_{k - 1} \times 〈 {α}^{\frac{1}{N}} 〉 ] & {ρ}_{1} \\ η & {ρ}_{2}\end{matrix} if ρ < 2$$

In the above equations, [symbol] x [symbol] implies that if x is not divisible by 4 for a quaternion case then x+(4−x % 4), η is the number of feature maps, ρᵢ indicates the i^{th} quaternion convolution layer in a residual block, N is the total number of residual units in the network. The only difference between these two configurations is that the additive-based configuration gradually increases the feature maps linearly, whereas the multiplication-based configuration rises geometrically. On the contrary, the configuration can be set such that the channel dimensions can be kept constant across the network.

As an example, to show the effectiveness of the hypercomplex model, a traditional convolution network architecture [Lim, B., Son, S., Kim, H., Nah, S., & Mu Lee, K. (2017). Enhanced deep residual networks for single image super-resolution. In Proceedings of the IEEE conference on computer vision and pattern recognition workshops (pp. 136-144)] was compared with a quaternion based EDSR model. The parameter meter budget was unaltered, i.e., if traditional convolution used 64 filters, the quaternion convolution also used 64 filters. This network was trained for 150 epochs (1000 iterations per epoch). Each iteration consisted of 16 batch size of 48×48 size input. The input for traditional CNN consisted of R, G, B channels, while the quaternion CNN consisted of GRAY, R, G, B channels. The number of training images was set to 800, and the testing data was 100 images.

TABLE 1

| | Number of parameters | | |
|---|---|---|---|
| Network Type | (in Million) | PSNR | SSIM |
| Traditional | 40.2 | 34.38 | 0.9649 |
| Hyper Complex | 10.1 | 34.05 | 0.9421 |

The mean PSNR values are shown for the testing datasets in Table 1. Higher PSNR and SSIM values represent better results, and one can observe that the hypercomplex network performs close to the traditional convolutions with four times fewer parameters.

According to some embodiments, the present disclosure includes food object detection using classical methods and/or traditional NN, alpha-trimmed NN, and/or hypercomplex NN. Once the preprocessing is executed, the following step comproses food object detection. This is a vital process in calorie measurement, shape classification, and quality sorting [Turgut, Sebahattin Serhat, Erkan Karacabey, and Erdo\check{g}an Küçüköner. “Potential of image analysis based systems in food quality assessments and classifications.” 9th Baltic Conference on Food Science and Technology “Food for Consumer Well-Being.” 2014.] This non-intrusive food recognition system relies on identifying unique features and pairing like features for identification and classification.

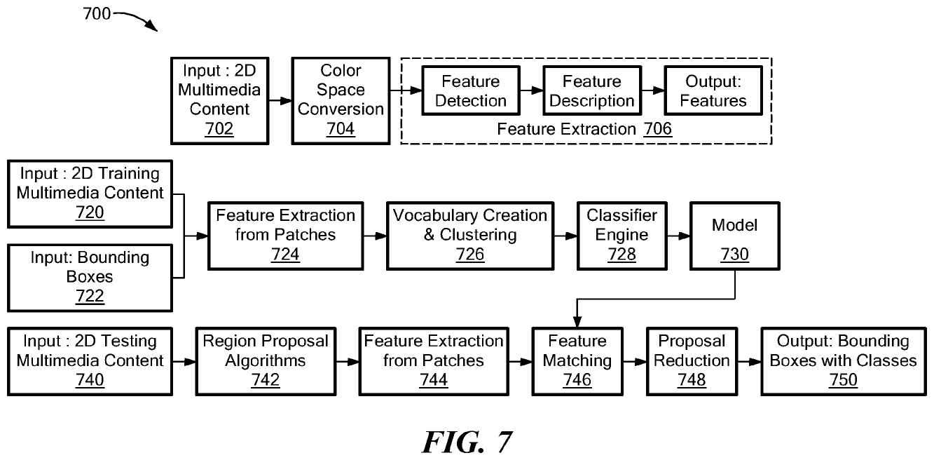

FIG. 7 illustrates a flowchart 700 of a classical object detection technique for 2D MC 702 with color space conversion 704 and feature extraction 706. In the case of classical object classification methodology, features such as corners, blobs, edges are extracted 702 using feature detectors. Various feature detectors have been proposed that include a Difference of Gaussian (DoG) detector (Lindeberg, Tony, “Feature Detection with Automatic Scale Selection,” International Journal of Computer Vision, 30.2: 79-116 (1998)), a Harris detector (Derpanis, Konstantinos G. “The harris corner detector.” York University (2004)), Binary Robust Independent Elementary Features (BRIEF) detector (Calonder, Michael, et al. “Brief: Binary robust independent elementary features.” European conference on computer vision. Springer, Berlin, Heidelberg, 2010), an Oriented FAST and Rotated BRIEF (ORB) detector (Rublee, Ethan, et al. “ORB: An efficient alternative to SIFT or SURF.” Computer Vision (ICCV), 2011 IEEE international conference on. IEEE, 2011), an AKAZE detector (Alcantarilla, Pablo F., and T. Solutions. “Fast explicit diffusion for accelerated features in nonlinear scale spaces.” IEEE Trans. Patt. Anal. Mach. Intell 34.7 (2011): 1281-1298), a Hessian detector, a Multiscale Hessian detector, a Hessian Laplace detector (“An Affine Invariant Interest Point Detector,” Computer Vision—ECCV 2002, 128-142 (2002)), a Harris Laplace detector, SURF (Bay, Herbert, Tinne Tuytelaars, and Luc Van Gool. “Surf: Speeded up robust features.” European conference on computer vision. Springer, Berlin, Heidelberg, 2006), and a Multiscale Harris detector. Furthermore, a comparative study of few of these detectors is provided in Agaian, Sos S. et al., “A Comparative Study of Image Feature Detection and Matching Algorithms for Touchless Fingerprint Systems,” Electronic Imaging, 2016.15: 1-9 (2016). Each detector identifies points uniquely and is invariant to various kinds of transformation.

In addition to the above-described detectors, a SIFT descriptor is commonly used in the field of computer vision. The SIFT descriptor was first presented by Lowe, David G. “Distinctive Image Features from Scale-Invariant Keypoints,” International Journal of Computer Vision, 60.2:91-110 (2004). SIFT uses a combination of the Difference of Gaussians (DoG) interest region detector and a corresponding feature descriptor to locate features in the image. This detector can be replaced by different detectors mentioned above, and they deliver a good performance. The feature vectors obtained from the detectors are uniquely making it invariant to complications such as rotation, translation, and object scaling. Additionally, feature descriptors without any description can also be provided. For example, a histogram of oriented gradients (HOG) as explained in Dalal, Navneet, and Bill Triggs. “Histograms of oriented gradients for human detection.” Computer Vision and Pattern Recognition, 2005. CVPR 2005. IEEE Computer Society Conference on. Vol. 1. IEEE, 2005. can also be employed. Feature extraction is either feature extraction w/ or w/o feature description.

During training 720, MC (from different views, and with eventual deformations) along with the bounding boxes 722 are given as input. These bounding boxes indicate the objects in the image and class of the image are attached to them. These bounding boxes are used to crop the MC, and features are extracted 724 using the aforementioned detectors. The next step is vocabulary creation 726 wherein features (points detected and/or described) are employed to construct vocabulary (dictionary of visual words) and represent each patch as a frequency histogram of features that are in the MC. These codewords are used to create clusters. Various clustering techniques include, but not limited to, kmeans (Arthur, David, and Sergei Vassilvitskii. “k-means++: The advantages of careful seeding.” Proceedings of the eighteenth annual ACM-SIAM symposium on Discrete algorithms. Society for Industrial and Applied Mathematics, 2007), mean shift (Comaniciu, Dorin, and Peter Meer. “Mean shift: A robust approach toward feature space analysis.” IEEE Transactions on pattern analysis and machine intelligence 24.5 (2002): 603-619), DBSCAN (Ester, Martin, et al. “A density-based algorithm for discovering clusters in large spatial databases with noise.” Kdd. Vol. 96. No. 34. 1996), Gaussian Mixture Model (Zivkovic, Zoran. “Improved adaptive Gaussian mixture model for background subtraction.” Pattern Recognition, 2004. ICPR 2004. Proceedings of the 17th International Conference on. Vol. 2. IEEE, 2004) can be employed. Next, the histograms of the visual words for each of the training patch in MC is aggregated. These are fed to a classifier 728 which provides an output to a model 730. The classifier may include Logistic Regression, Naive Bayes Classifier, Support Vector Machines, Decision Trees, Boosted Trees, Boosted Trees, Boosted Trees, Boosted Trees, etc. These classifiers can also be combined using ensemble methods. These improve generalizability/robustness over a single estimator by combining the predictions of several base estimators built with a given learning algorithm. For example, bagging methods, the forest of randomized trees, AdaBoost, Gradient Tree Boosting, etc. During the training phase, the classifier tries to classify different classes depending on the vocabulary.

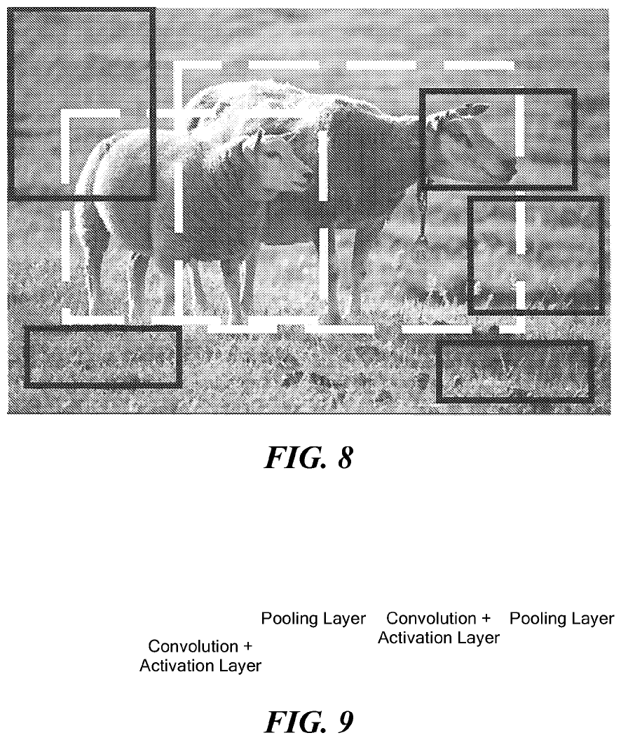

During a testing phase 740, MC without bounding boxes is given as input. Next, sliding window processing 742 can be applied to generate smaller sub-regions/patches of multiple objects. Even though this approach is less complex, it has a high time complexity. Alternatively, region proposal algorithms 742 can be used. These methods take MC and provide bounding boxes corresponding to all patches that are most likely to be objects. These proposed regions can be noisy, overlapping, and may not contain an object perfectly. Example region proposal algorithms are Objectness as described in Alexe, Bogdan, Thomas Deselaers, and Vittorio Ferrari. “Measuring the objectness of image windows.” IEEE transactions on pattern analysis and machine intelligence 34.11 (2012): 2189-2202, Constrained Parametric Min-Cuts for Automatic Object Segmentation as described in Carreira, Joao, and Cristian Sminchisescu. “Constrained parametric min-cuts for automatic object segmentation.” Computer Vision and Pattern Recognition (CVPR), 2010 IEEE Conference on. IEEE, 2010, Category Independent Object Proposals as described in Endres, Ian, and Derek Hoiem. “Category independent object proposals.” European Conference on Computer Vision. Springer, Berlin, Heidelberg, 2010, Randomized Prim as described in Manen, Santiago, Matthieu Guillaumin, and Luc Van Gool. “Prime object proposals with randomized prim's algorithm.” Proceedings of the IEEE international conference on computer vision. 2013, and Selective Search as described in Uijlings, Jasper R R, et al. “Selective search for object recognition.” International journal of computer vision 104.2 (2013): 154-171. These region proposal methods provide probability scores, and the patch with a high probability score are locations of the objects. An example of the region proposal algorithm can be seen in FIG. 8, where the dotted lines are the patches where objects exist, and the solid boxes are noisy patches where objects do not exist. After the region proposal, MC in the proposed region is considered for feature detection and description. The features extracted 744 are then matched/classified 746 using the trained classifier as region proposal technique provides multiple bounding boxes that are overlapping and duplicates. To remove overlapping boxes 748, method such as, but not limited to non-maximum suppression can be used. Finally, the output 750 comprises MC with bounding boxes around the object and class of the object.

According to some embodiments, the present disclosure includes systems and methods for object detection using deep learning architectures. In contrast to classical classification techniques, deep convolutional neural networks that combine both feature extraction and classification can also be employed. These networks can be trained end-to-end from the MC to the corresponding labels and bounding boxes.

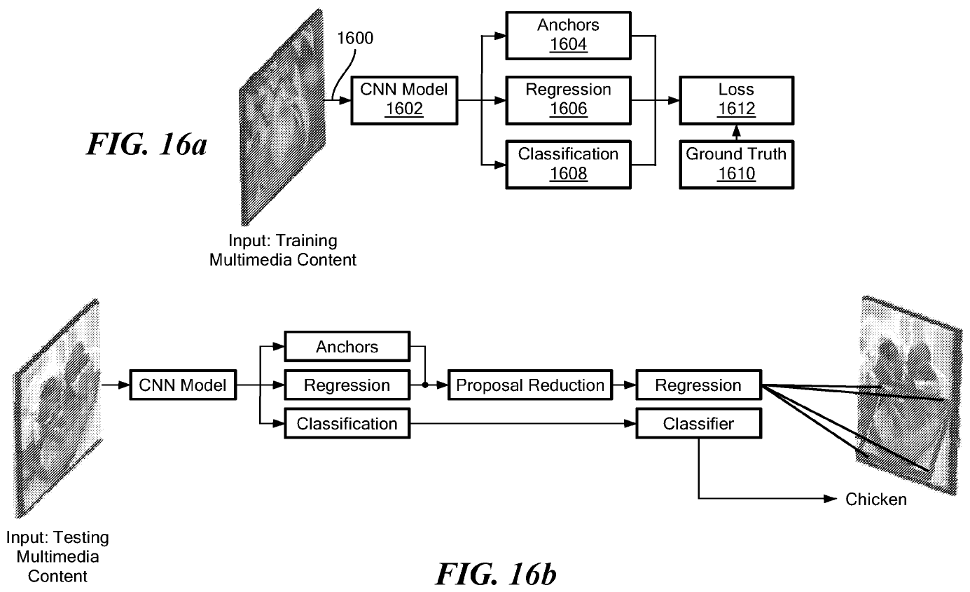

A general flowchart for training and testing can be visualized in FIG. 16a and FIG. 16b, respectively. During the training phase, multimedia content is provided as input 1600 to the CNN model 1602 or a hypercomplex model or an alpha trimmed model, which generates information for anchor 1604, regression 1606, and classification 1608 processing. These outputs and ground truth 1610 can be provided to a loss module 1612. An example of the CNN model is displayed in FIG. 9. The aforementioned layers, such as traditional, hypercomplex, alpha-trimmed convolution, activation, normalization, and pooling layers, can be stacked in any fashion. These aid in computing a feature map over an entire input image. These features include generalizable and abstract features by hierarchical representation, e.g., CNNs trained on ImageNet database can extract eyes, tail, etc., as features in their last layers. These perform feature extraction, which will be used within the neural network architecture and are end-to-end trainable. This will be named as the backbone network in the entirety. For object region proposals, the aforementioned techniques can be used. Along with those object region proposal methods, another method named anchors were introduced in Ren, Shaoqing, et al. “Faster r-cnn: Towards real-time object detection with region proposal networks.” Advances in neural information processing systems. 2015, can also be employed. every sliding window center creates fixed k anchor boxes and is associated with a scale and an aspect ratio.

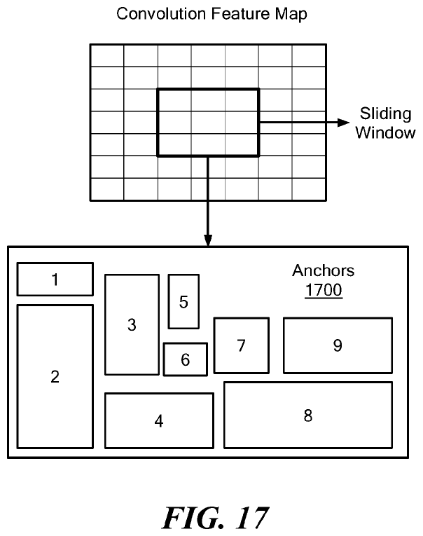

An example of the anchor creation is shown FIG. 17. These anchors 1700 are translation invariant, both in terms of the anchors and the functions that compute proposals relative to the anchors.

The features extracted from the backbone network are fed to the regression 1606 and classification 1608 model. The regression and/or classification models 1606, 1608 comprise the aforementioned CNN layers stacked in any fashion. The regression model 1606 returns a number; in this case, it returns the coordinates of the predicted bounding box. These models are generally a small fully connected layer, or a convolution layer strides through this feature map, and at each location, it can predict the x position, y position, height of the box, width of the box values for each anchor boxes. For example, if a feature map of size 50×50 is provided and the number of anchors is 9, the output of the convolution layer is 50×50×9×4. Similarly, the classification model predicts the probability of an object present art each location of each anchor box. For example, if a feature map of size 50×50 is provided and the number of anchors is 9, the output of the is 2500×9. The feature map created may have a loss in semantic information at a low level due to subsampling processes. This lowers the ability to detect small objects in the image.

As a result, feature pyramid networks can be used as explained in Lin, Tsung-Yi, et al. “Focal loss for dense object detection.” IEEE transactions on pattern analysis and machine intelligence (2018). This technique constructs a rich, multi-scale feature pyramid from a single resolution input image by augmenting a convolutional network with a top-down pathway. Furthermore, each level in this pyramid can be used to detect objects that can be found on different scales. For food detection and classification purposes, the input these networks can comprise food images with bounding boxes and food class defined by humans. A network can be designed with the aforementioned layers and trained in an end to end fashion. The object detection training time can be lowered by first training the said designed backbone network as a classification network using large datasets. Once the models have converged, these can be reutilized as a backbone for object detection and fine-tuned for food datasets. While testing the models, only the food images will be provided as input, and the model provides the location and the food class. As many anchors are present, the number of bounding boxes is higher. To reduce this method, which includes but not limited to, non-maximal suppression can be used.

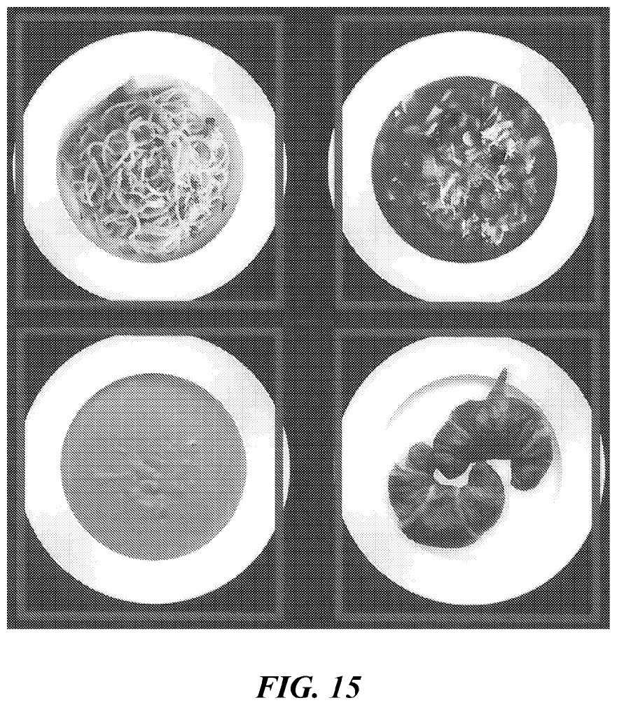

An example output of the model can be visualized in FIG. 15. The loss function 1612 may include functions proposed in Ren, Shaoqing, et al. “Faster r-cnn: Towards real-time object detection with region proposal networks.” Advances in neural information processing systems. 2015, Redmon, Joseph, et al. “You only look once: Unified, real-time object detection.” Proceedings of the IEEE conference on computer vision and pattern recognition. 2016, Lin, Tsung-Yi, et al. “Focal loss for dense object detection.” IEEE transactions on pattern analysis and machine intelligence (2018), Nercessian, Shahan, Sos S. Agaian, and Karen A. Panetta. “An image similarity measure using enhanced human visual system characteristics.” Mobile Multimedia/Image Processing, Security, and Applications 2011. Vol. 8063. International Society for Optics and Photonics, 2011, Silva, Eric A., Karen Panetta, and Sos S. Agaian. “Quantifying image similarity using measure of enhancement by entropy.” Mobile Multimedia/Image Processing for Military and Security Applications 2007. Vol. 6579. International Society for Optics and Photonics, 2007, Agaian, Sos S. “Visual morphology.” Nonlinear Image Processing X. Vol. 3646. International Society for Optics and Photonics, 1999. Panetta, Karen A., Eric J. Wharton, and Sos S. Agaian. “Human visual system-based image enhancement and logarithmic contrast measure.” IEEE Transactions on Systems, Man, and Cybernetics, Part B (Cybernetics) 38.1 (2008): 174-188.

According to some embodiments, the present disclosure includes systems and methods for food segmentation using deep learning techniques. Semantic segmentation is the pixel-wise labeling of an image. Since the problem is defined at the pixel level, pixel resolution is necessary to localize them at the original image. In the case of food segmentation, pixel-level classification is required to determine which food item is in the image.

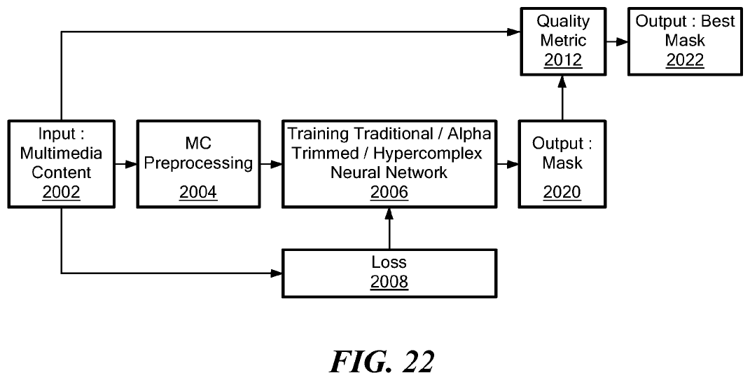

An example of the flowchart for training deep learning-based super-resolution networks is provided in FIG. 22, which may have some similarity to FIG. 20. The input to the system depends on the end needs. For example, for food categories, Gray+R+G+B channels can be employed, for food freshness, hyperspectral, or thermal, or a combination of these can be utilized to define a mask 2022 to figure out places where the food is spoilt, etc. The preprocessing may include but is not limited to the aforementioned steps such as filtering, enhancement, etc. As an example, the hypercomplex-based quaternion layer is utilized for description purposes.

An illustrative example of a possible architecture that can be utilized for generating segmentation masks can be visualized in FIG. 23a. The architecture includes an encoder and decoder architecture. The encoder architecture aims at generating a high-dimensional global quaternion feature vector. In contrast, the decoder architecture utilizes the extracted high-dimensional global quaternion feature vector and generates a semantic segmentation mask. The encoder architecture can utilize the same backbone, or a new neural network architecture can be constructed utilizing the aforementioned layers. The backbone may comprise of T/A/H blocks stacked serially or in a parallel fashion. For segmentation purposes, global information is required to segment different regions in the provided MC effectively. To perform this, the feature maps need to be downsampled after a certain number of T/A/H blocks to obtain global features. This can be visualized in the encoder section in FIG. 23a, where the feature maps are extracted and downsampled by a factor of 2 to encapsulate different regions in the MC. The T/A/H blocks may comprise the layers visualized in FIG. 21c and/or FIG. 23b. The number of feature maps in each block can be defined using Equation 24 and/or Equation 25. The network may comprise a fusion layer that combines the global features obtained from a downsampled feature space and an intermediate layer feature space. During the decoding phase, the feature space is upsampled by a factor of 2 until the original MC resolution is recovered. The feature space from the corresponding encoding phase is combined during the upsampling process to provide the extracted features obtained from the encoding phase. Finally, the result is a semantic segmentation mask. The loss function may comprise cross-entropy loss, L1 loss, focal loss, or any of the loss functions mentioned in [Minaee, S., Boykov, Y., Porikli, F., Plaza, A., Kehtarnavaz, N., & Terzopoulos, D. (2020). Image Segmentation Using Deep Learning: A Survey. arXiv preprint arXiv:2001.05566.]. Alternately, the method proposed by Panetta et al [Panetta, K., Kamath, K. S., Rajeev, S., & Agaian, S. S. (2021). FTNet: Feature Transverse Network for Thermal Image Semantic Segmentation. IEEE Access, 9, 145212-145227.] can also be utilized to perform semantic segmentation. The same can be extended to hypercomplex space to perform segmentation efficiently.

As an example, to show the effectiveness of the hypercomplex model, a traditional convolution network architectures [Zhao, H., Qi, X., Shen, X., Shi, J., & Jia, J. (2018). Icnet for real-time semantic segmentation on high-resolution images. In Proceedings of the European Conference on Computer Vision (ECCV) (pp. 405-420). And A. Greenblatt, S. Agaian, Introducing quaternion multi-valued neural networks with numerical examples, Information Sciences Volume 423, January 2018, Pages 326-342] was compared with a quaternion-based ICNET model. ICNET comprises of an image cascade network that incorporates multi-resolution branches under proper label guidance to reduce a large portion of computation for pixel-wise label inference. For comparison purposes, a deep ResNet 50 model defined in the paper was utilized as a backbone. The parameter meter budget was unaltered i.e., if traditional convolution used 64 filters, the quaternion convolution also used 64 filters. This network was trained on the UNIMIB 2016 [Ciocca, G., Napoletano, P., & Schettini, R. (2016). Food recognition: a new dataset, experiments, and results. IEEE journal of biomedical and health informatics, 21(3), 588-598.] dataset for 400 epochs. Each iteration consisted of 3 batch sizes of multiscale input, i.e., the network was randomly fed with different input resolutions. The input images were cropped to scale with a long side of either 704 or 480 size. The input for traditional CNN consisted of R, G, B channels, while the quaternion CNN consisted of GRAY, R, G, B channels. The number of training images was 650, and the testing data was 360 images.

TABLE 2

| | Number of parameters | Pixel | Mean |
|---|---|---|---|
| Network Type | (in Million) | Accuracy | IOU |
| Traditional | 28.29 | 90.6% | 39.90% |
| Hyper Complex | 8.071 | 90.9% | 41.76% |

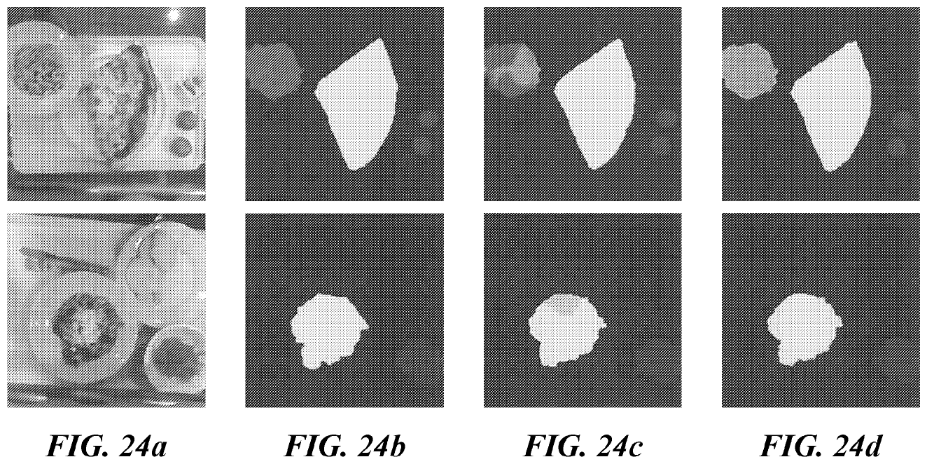

The mean IOU values are shown for the testing datasets in Table 2. Higher MIOU and Pixel Accuracy values represent better results, and one can observe that the hypercomplex network outperforms traditional convolutions with four times fewer parameters. FIGS. 24a-d provides a visual comparison between these networks. It can be seen that conventional CNNs fail in segmenting a few food items while hypercomplex CNNs contain better segments.

According to some embodiments, the present disclosure includes systems and methods for three-dimensional multimedia content reconstruction using traditional techniques is provided. In computer vision, various approaches exist to reconstruct 3-D models. Methods to obtain 3-D models of the food objects can include but are not limited to Sagawa, Ryusuke, et al. “Dense one-shot 3D reconstruction by detecting continuous regions with parallel line projection.” Computer Vision (ICCV), 2011 IEEE International Conference on. IEEE, 2011, Kanade, Takeo, and Masatoshi Okutomi. “A stereo matching algorithm with an adaptive window: Theory and experiment.” IEEE transactions on pattern analysis and machine intelligence 16.9 (1994): 920-932, Woodham, Robert J. “Photometric stereo: A reflectance map technique for determining surface orientation from image intensity.” Image Understanding Systems and Industrial Applications I. Vol. 155. International Society for Optics and Photonics, 1979, Usamentiaga, Ruben, Julio Molleda, and Daniel F. Garcia. “Fast and robust laser stripe extraction for 3D reconstruction in industrial environments.” Machine Vision and Applications 23.1 (2012): 179-196, Lanman, Douglas, and Gabriel Taubin. “Build your own 3D scanner: optical triangulation for beginners.” ACM SIGGRAPH ASIA 2009 Courses. ACM, 2009, Huang, Peisen S., and Song Zhang. “Fast three-step phase-shifting algorithm.” Applied optics 45.21 (2006): 5086-5091. Alternatively, Structure from Motion (SfM) can also be used for the 3-D reconstruction of food objects.

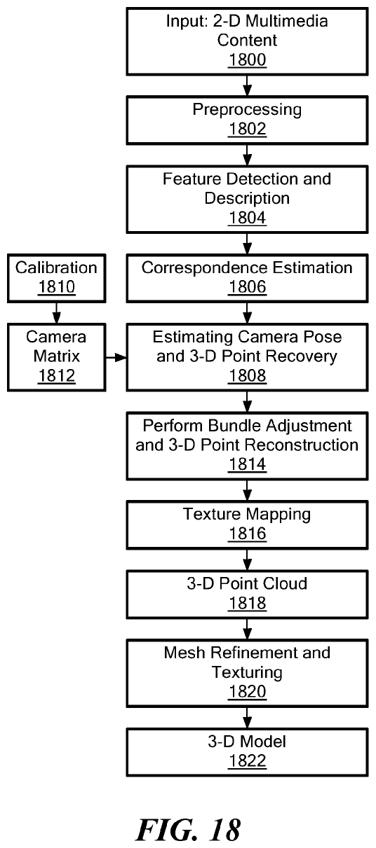

An example flow chart of the 3-D reconstruction method is shown in FIG. 18. The input 1800 to the pipeline is multimedia content (images/videos from different sensors). An example of the side view and top view data acquisition process is shown in FIG. 5a and FIG. 5b, respectively. In step 1802, preprocessing techniques, for example, filtering, recoloring, color space conversion (as mentioned before), etc., are applied.

After preprocessing, feature detection and description processing 1804 are employed to detect features and their description in each multimedia content. Correspondence estimation processing is performed in step 1806 and can include a) feature matching and/or b) feature tracking. In the feature matching algorithm, definitive correspondence between the features is computed. This helps remove the geometrically inconsistent outliers and provides a relative pose considering all pair-wise configurations. In feature tracking, an algorithm is employed to trace a query feature and over-impose it in the following images, thus creating a track for each particular feature across all the provided multimedia content. In step 1808, camera pose and 3D point recovery are estimated after calibration 1810 and camera matrix processing 1812.

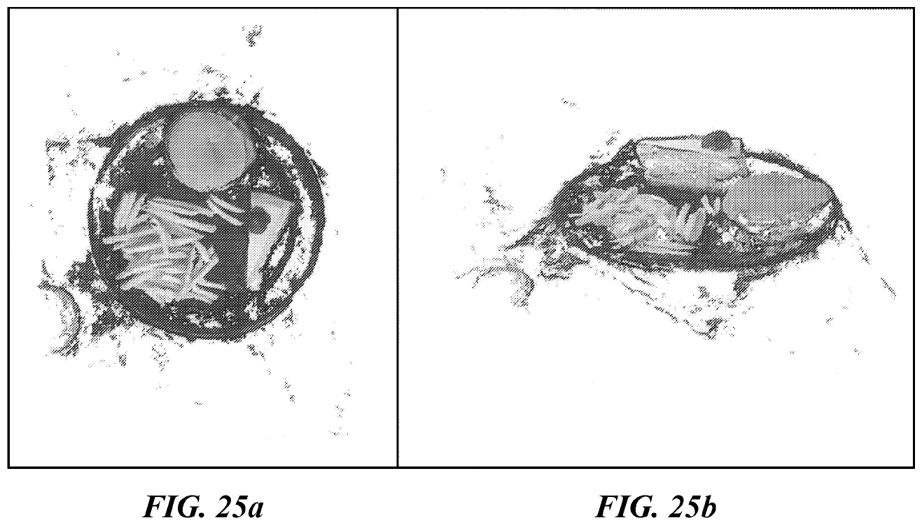

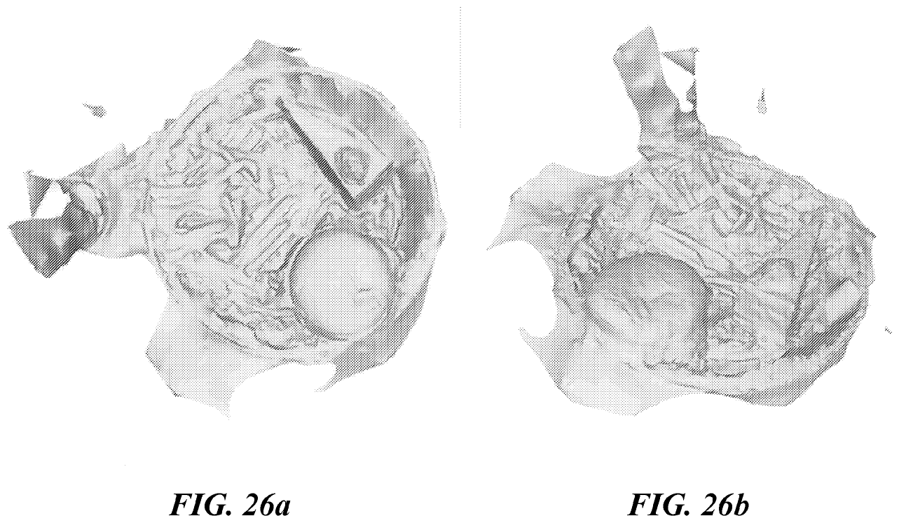

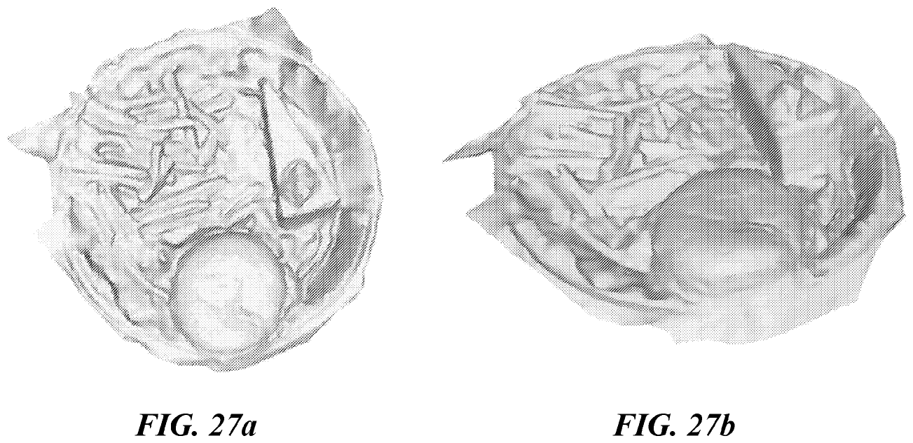

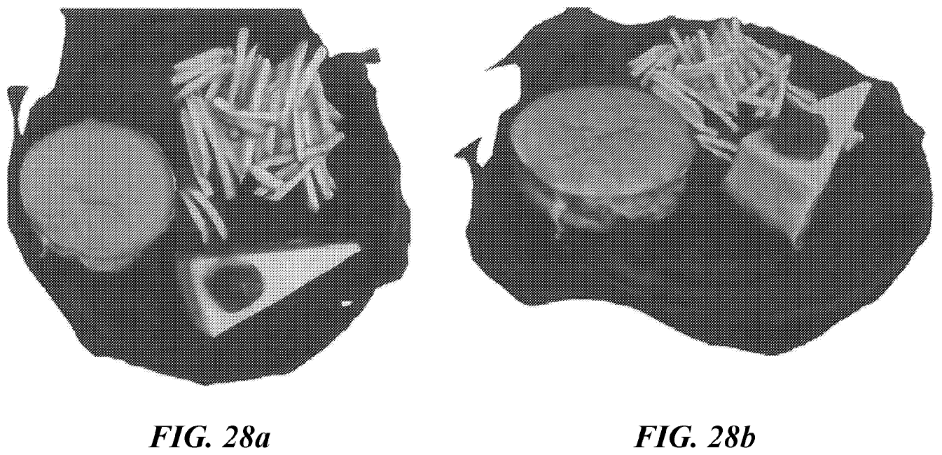

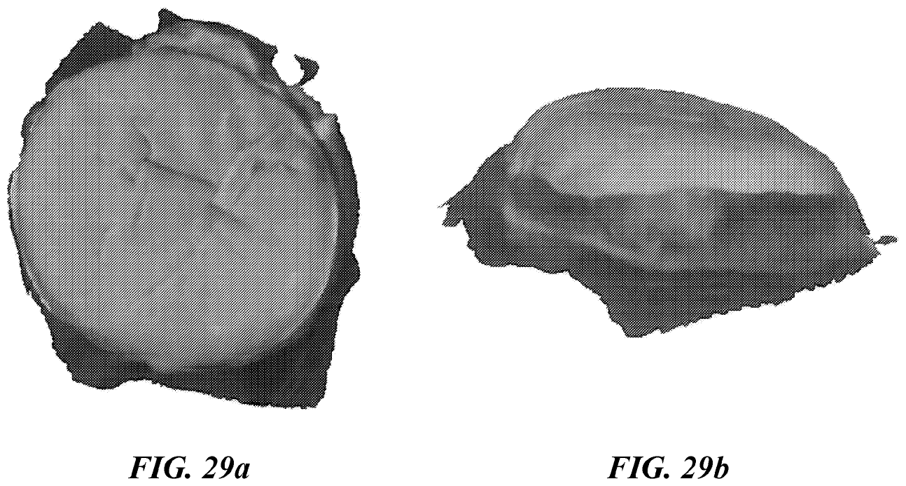

In step 1814, bundle adjustment (BA) is performed to jointly optimize camera and point parameters and when a new pose is added. As a new pose gets added, new geometrically valid 3-D points are also added. BA is used to minimize the reprojection error by refining parameters and flushing out bad points. The 3-D point cloud 1818 is obtained without any texture; texture mapping is performed in step 1816 to map color onto the points. An example of the 3-D point cloud obtained using images from FIG. 6 can be seen in FIG. 19 as it can be noticed that the point clouds obtained have holes and is a typical sparse point-cloud. In step 1820, a mesh is reconstructed to generate a 3D model in step 1822. To construct a mesh, any of the methods explained in Berger, Matthew, et al. “State of the art in surface reconstruction from point clouds.” EUROGRAPHICS star reports. Vol. 1. No. 1. 2014. can be used. Alternatively, hole filling algorithms as surveyed in “Guo, X., Xiao, J., & Wang, Y. (2018). A survey on algorithms of hole filling in 3D surface reconstruction. The Visual Computer, 34(1), 93-103.” can also be used to reconstruct the point data lost due to occlusion, reflectance, the scanning angle, and raw data preprocessing. An example of the reconstructed mesh can be visualized in FIG. 25 (a) (top view) and FIG. 25 (b) (side view). The mesh obtained either from the sparse or dense point-cloud can be further refined to recover all fine details or even bigger missing parts. The refined mesh with finer details can be visualized in FIG. 26a (top view) and FIG. 26b (side view). Finally, the texture is mapped to these meshes using the multimedia content and can be visualized in FIG. 28a (top view) and FIG. 28b (side view). These 3-D food items are then localized using the bounding boxes computed in the object detection and recognition step. An example of a food item extracted from the 3-D model can be seen in FIG. 29a (top view) and FIG. 29b (side view).

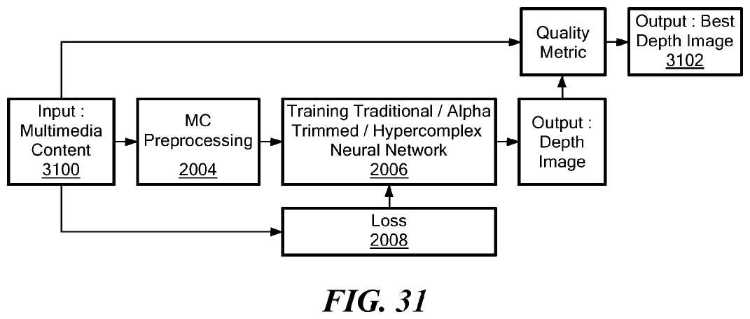

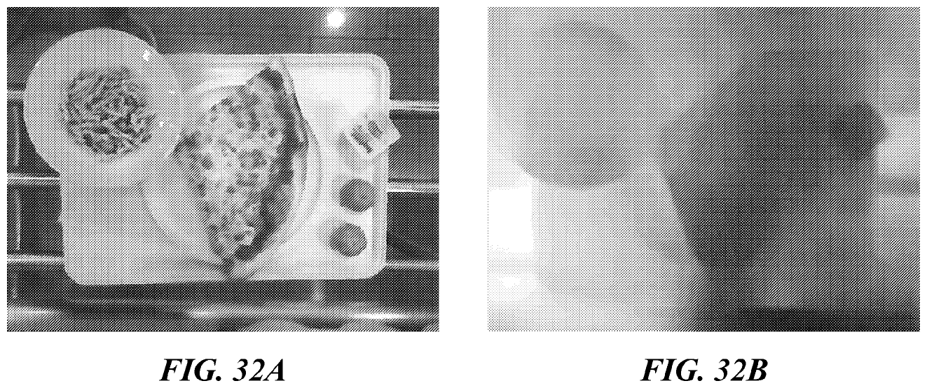

According to some embodiments, the present disclosure includes systems and methods for three-dimensional multimedia content reconstruction using deep learning techniques is provided, such as that shown in FIG. 31, which may be similar to FIG. 20. FIG. 31 receives an MC as input 3100 and the output is a depth map 3102. It may utilize the H/A/T layer to define a neural network. Reconstruction of a depth map can be considered to be similar to semantic segmentation. The only difference is that semantic segmentation is trained against a mask while the depth map reconstruction is trained against a depth map. Thus, most of the structure may follow the semantic segmentation architecture described before, or a new neural network can be formed. The networks and loss functions may also comprise of any of the methods provided in [Bhoi, A. (2019). Monocular depth estimation: A survey. arXiv preprint arXiv:1901.09402, or Laga, H. (2019). A Survey on Deep Learning Architectures for Image-based Depth Reconstruction. arXiv preprint arXiv:1906.06113.]. An example of the depth map reconstructed using [Hu, J., Ozay, M., Zhang, Y., & Okatani, T. (2019, January). Revisiting single image depth estimation: Toward higher resolution maps with accurate object boundaries. In 2019 IEEE Winter Conference on Applications of Computer Vision (WACV) (pp. 1043-1051). IEEE.] is displayed in FIG. 32.

According to some embodiments, the present disclosure includes systems and methods for three-dimensional multimedia content volume estimation. The 3-D meshes obtained from the previous step is a structural build of a 3-D model comprising polygons. 3-D meshes use reference points in X, Y, and Z coordinates to define shapes with height, width, and depth. The polygons generally consist of quadrangles, triangles which can be further broken down into vertices in X, Y and Z coordinates and lines.

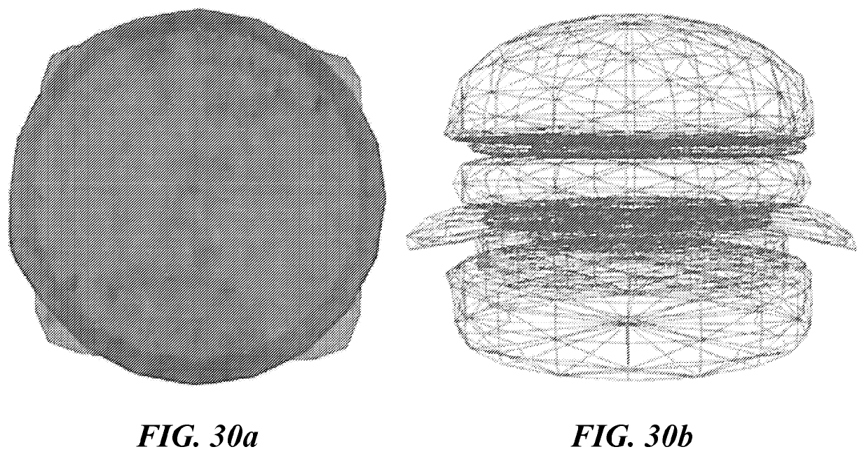

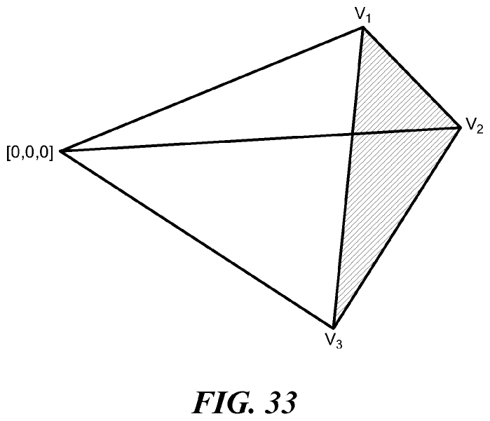

An example of the polygons generated can be visualized in FIG. 30a (top view with color and mesh) and FIG. 30b (side view of just the mesh). For simplicity of exposition, consider the polygons to be a triangle. The extension to different polygons is straightforward. The triangles are two-dimensional and thus do not have any volume metric associated. These triangles are converted into a tetrahedron, which starts from the origin [0,0,0] to the triangle. An example of the tetrahedron is provided in FIG. 33. Let the vertices of the triangle be denoted by V₁=[v₁₁, v₁₂, v₁₃], V₂=[v₂₁, v₂₂, v₂₃] and V₃=[v₃₁, v₃₂, v₃₃]. The volume of the tetrahedron can be computed as formulated in Equation 26 and Equation 27.

$${Volume}_{tetrahedron} = \frac{1}{6} [ {V}_{1} \times {V}_{2} ] \cdot {V}_{3} \qquad Equation 26$$

$${Volume}_{tetrahedron} = \frac{1}{6} [ {v}_{11} , {v}_{22} , {v}_{33} + {v}_{12} , {v}_{23} , {v}_{31} + {v}_{13} , {v}_{21} , {v}_{32} - {v}_{11} , {v}_{23} , {v}_{32} - {v}_{12} , {v}_{21} , {v}_{33} - {v}_{13} , {v}_{22} , {v}_{31} ] \qquad Equation 27$$

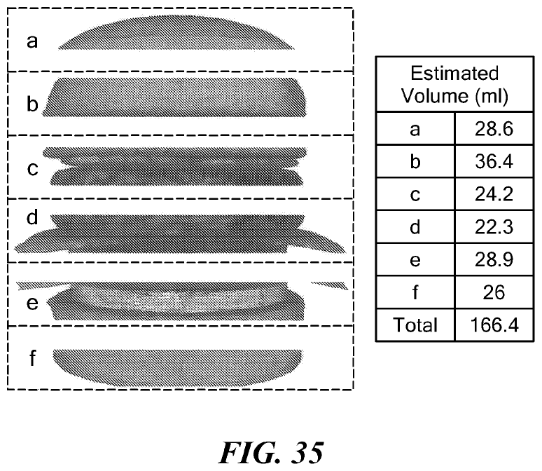

Alternatively, for irregular or partial objects, the organized mesh can be divided into slices, as visualized in FIG. 35. In particular, the object in an organized point cloud can use each row as a slice. The slice thickness can be computed by taking the difference between maximum and minimum horizontal or vertical values (h/v) and dividing it by the number of rows/columns. Before dividing the point cloud into slices, certain preprocessing needs to be applied in order to remove noise. For example, a median filter can be applied along the x, y, z directions to reduce noise. Alternately a histogram filter can be applied to remove outliers. The histogram filter uses a histogram with n-bins and tries to find bins with the highest frequency values. Then the highest bin residing below the peak with 5% of the peak frequency and the lowest bin residing above the 5% of the peak frequency is determined. These values corresponding to these bins are considered to the minimum and maximum horizontal or vertical values allowable values. This aids in conveniently performing volume estimation without the need to rearrange the objects while scanning. W

The slice width can be computed using the following.

$${w}_{s l i c e} = \frac{\begin{matrix}Maximum along h ⋰ v - \\ Minumum along h ⋰ v\end{matrix}}{{N}_{s l i c e s}} \qquad Equation 28$$

where maximum or minimum along horizontal or vertical values is found using the histogram filter and N_{slices} is the number of slices to be considered.

Once the slices are obtained conic sections-based fitting can be employed. Few of the conic sections include but are not limited to ellipse, circles, parabolas, and hyperbolas. A general equation that describes the conic section is provided below:

Ax²+2Bxy+Cy²+2Dx+2Ey+F=0 Equation 29

where (x,y) are the points of the conic, A, B, C, D, E, and F are implicit parameters and are used to infer the shape of the conic. For example, to obtain an ellipse, the requirement is that B²−4AC also known as discriminant, should be negative. In another example, to obtain a circle, the requirement is that A=C and B=0. For explanation purposes, circle and elliptical fitting is provided.

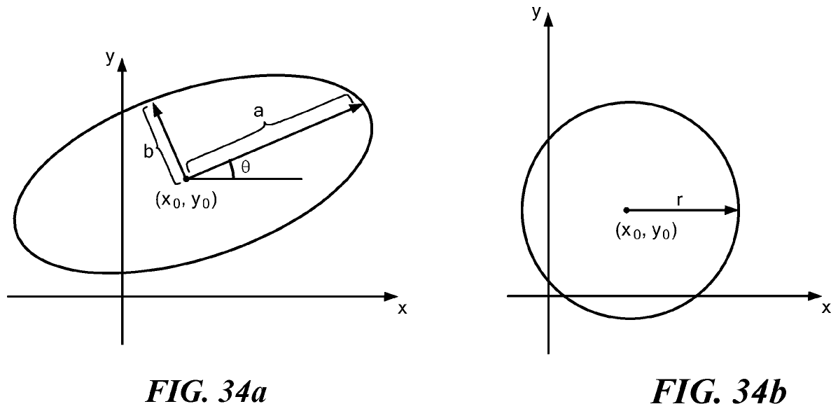

An ellipse defined by a center (x₀, y₀), a semi-major axis a, a semi-minor axis b, and an angle θ can be visualized in FIG. 34a. Canonically, an ellipse can be written as Equation 30. The relation between (x′, y′) and (x,y) can be formulated as Equation 31 and the area is given by Equation 32.

$$\frac{{( {x}^{′} - {x}_{0}^{′} )}^{2}}{{a}^{2}} + \frac{{( {y}^{′} - {y}_{0}^{′} )}^{2}}{{b}^{2}} = 1 \qquad Equation 30$$

$$where ( {x}^{′} , {y}^{′} ) and ( {x}_{0}^{′} , {y}_{0}^{′} ) are the coordinates \\ when an angle θ exists \\ between ( x , y ) and ( {x}_{0} , {y}_{0} ) around the origin$$

$$[ \begin{matrix}x \\ y\end{matrix} ] = [ \begin{matrix}cos θ & - sin θ \\ sin θ & cos θ\end{matrix} ] [ \begin{matrix}{x}^{′} \\ {y}^{′}\end{matrix} ] \qquad Equation 31$$

$${Area}_{e l l i p s e} = π a b \qquad Equation 32$$

As volume estimation requires area, it is necessary to compute the semi-major axis and semi-minor axis lengths from the canonical form in Equation 29. The equations for semi-major axis a, a semi-minor axis b can be represented as shown below:

$$a = \sqrt{\frac{2 ( {AE}^{2} + {CD}^{2} + {FB}^{2} - 2 BDE - 2 CF )}{- ( {B}^{2} - A C ) [ \sqrt{{( A - C )}^{2} + 4 {B}^{2}} - ( A + C ) ]}} \qquad Equation 33$$

$$b = \sqrt{\frac{2 ( {AE}^{2} + {CD}^{2} + {FB}^{2} - 2 BDE - 2 CF )}{- ( {B}^{2} - A C ) [ - \sqrt{{( A - C )}^{2} + 4 {B}^{2}} - ( A + C ) ]}} \qquad Equation 34$$

A circle is an ellipse with a semi-major axis equal to the semi-minor axis. As the shape is symmetrical, 0 is irrelevant. A circle with a center (x₀, y₀) and radius r can be visualized in FIG. 34b. The canonical form and the area can be found using Equation 35 and Equation 36, respectively.

$$\frac{{( {x}^{′} - {x}_{0}^{′} )}^{2}}{{r}^{2}} + \frac{{( {y}^{′} - {y}_{0}^{′} )}^{2}}{{r}^{2}} = 1 \qquad Equation 35$$

$$where ( {x}^{′} , {y}^{′} ) and ( {x}_{0}^{′} , {y}_{0}^{′} ) are the coordinates \\ when an angle θ exists \\ between ( x , y ) and ( {x}_{0} , {y}_{0} ) around the origin$$

$${Area}_{e l l i p s e} = π {r}^{2} \qquad Equation 36$$

The radius r for circle fitting can be simplified using Equation 29 as follow:

$$r = \sqrt{\frac{A E - {E}^{2} - {D}^{2}}{{A}^{2}}} \qquad Equation 37$$

The algorithm fits the 2-D ellipse/circle to the points in each slice and computes the area of the ellipse/circle. The fitting algorithms can comprise of any methods described in “Kanatani, Kenichi, Yasuyuki Sugaya, and Yasushi Kanazawa. “Ellipse fitting for computer vision: implementation and applications.” Synthesis Lectures on Computer Vision 6.1 (2016): 1-141.”.

Then each slice thickness is multiplied with the area of each ellipse/circle and summed up together to estimate the final volume. This can be formulated, as shown in Equation 38.

$$Volume = {\sum }_{i = 0}^{number of slices} {Volume}_{i} \times {thickness}_{i} \qquad Equation 38$$

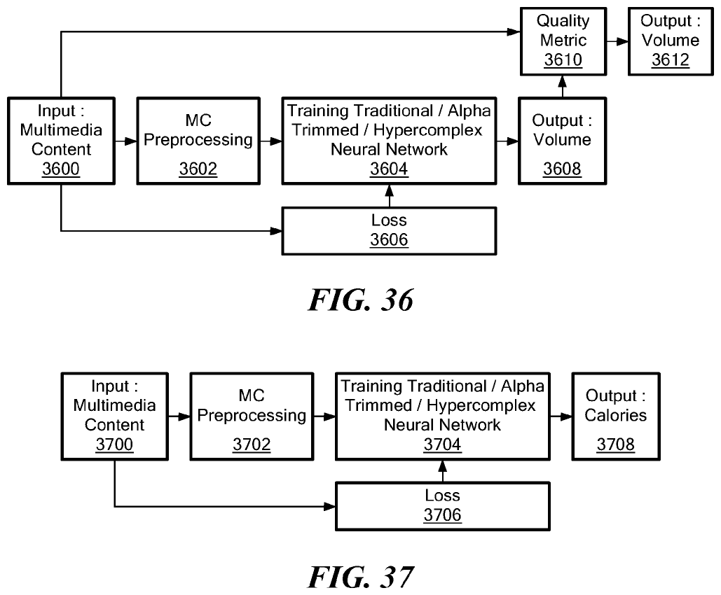

FIG. 36 is a flowchart of volume estimation using a deep learning method. The input 3600 to the system can include any kind of multimedia content. These can be preprocessed 3602 using the aforementioned techniques. For example, if the input is noisy, noise removal can be applied, if the input is of low quality, image enhancement can be applied. For training 3604 a network, ground truth calorie is required. A loss module 3606 can provide information to the training 3604 for generating a volume output 3608. MC input 3600 can be provided to a quality metric 3610, which receives the output 3608 and generates an enhanced volume output 3612

According to some embodiments, the present disclosure includes systems and methods for providing calorie estimation using the MC content. A flow chart for deep learning-based calorie estimation is illustrated in FIG. 37. The input 3700 to the system can include any kind of multimedia content. These can be preprocessed 3702 using the aforementioned techniques. For example, noise removal can be applied; if the input is of low quality, image enhancement can be applied. For training 3704 a network, ground truth calorie is required. A loss module 3706 can provide information to the training 3704 for generating a calorie output 3708. Ground truth calories can be computed using the weighted plate waste method. In this method, food items (i.e., entrees and sides, including condiments and sauces) are collected, including uneaten foods or empty packaging from fully consumed foods. Food items are weighed separately, in duplicate, to the nearest gram, using food scales and the average of the 2 measurements are calculated and recorded as the “plate waste weight” of the item. The item's weight is then converted to energy (kcal) using restaurant-stated pre-consumption weight and energy information.

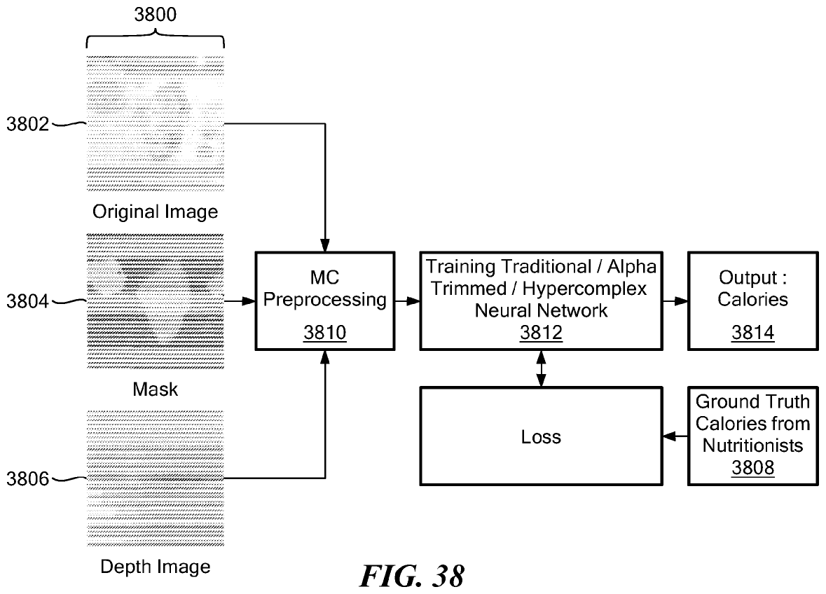

Alternatively, standard bomb calorimetry procedures can also be considered to compute the energy. This energy can be used as ground truth and used for training a neural network. An example of this system can be visualized in FIG. 38. The inputs 3800 can comprise a visible image 3802 along with a mask 3804 generated using segmentation and a depth image 3806 generated using depth map estimation techniques. The ground truth calories 3808 can be computed, as described above, using plate weight estimation or calorimetric bomb method, for example. The input 3800 can be preprocessed 3810 for a training process 3812 to output calories 3814.

According to some embodiments, the present disclosure includes systems and methods for providing calorie estimation using preexisting databases. The entire procedure is executed on multimedia content before (MCB) and after (MCA) food consumption. For each food item, the difference in volume between these MCB and MCA food consumption will be computed. This estimated volume of the consumed food is mapped to food weight estimation and linked to nutrient databases to determine nutrition-related data (e.g., calories, nutrients). The standard source for this is the USDA National Nutrient Database (NNDB) and the USDA Food and Nutrient Database for Dietary Studies (FNDDS) [Food Composition Databases Show Foods List. 2018; https://ndb.nal.usda.gov/ndb/; https://www.ars.usda.gov/northeast-area/beltsville-md-bhnrc/beltsville-human-nutrition-research-center/food-surveys-research-group/docs/fndds-download-databases/.]. Once, the automated system estimates the food type and nutrient content in the image and saves the information, further validation is conducted using gold standard techniques such as weighed plate waste and bomb calorimetry. This control mechanism guarantees the accuracy of the data to be used in food intake analysis. Also, once the food consumed is computed, the food waste can also be calculated using Equation 39.

Food wasted=MCB−(MCB−MCA) Equation 39

According to some embodiments, the present disclosure includes systems and methods for providing calorie estimation using deep learning methods. The input to these deep learning techniques may comprise a multimedia content or a combination of multimedia content. For example, the input to the system may include a set of images/videos before and after food consumption. The output of this system may comprise the calories consumed.

To generate ground truth, weighed plate waste methodology or bomb calorimetric techniques can be utilized to evaluate the calories, specifically the total grams, energy, and nutrient estimates. The percent of total grams consumed can be calculated by: (volume remaining x estimated density in grams per ml). Specific energy, macro- and micronutrients investigated will be total energy, macronutrients, and nutrients of concern according to the Scientific Report of the 2015 Dietary Guidelines Advisory Committee (i.e., calcium, fiber, iron, sodium, and saturated fat); and sugar. [https://health.gov/our-work/food-nutrition/2015-2020-dietary-guidelines]. The system can be trained by providing the MC as input and training against the generated ground truth. Finally, while testing, only the MC can be utilized to generate the expected output. For feature extraction, the encoder part of the segmentation or depth map reconstruction can be used. It helps in extracting the feature maps, and due to downsampling, global features can aid in providing the calories.

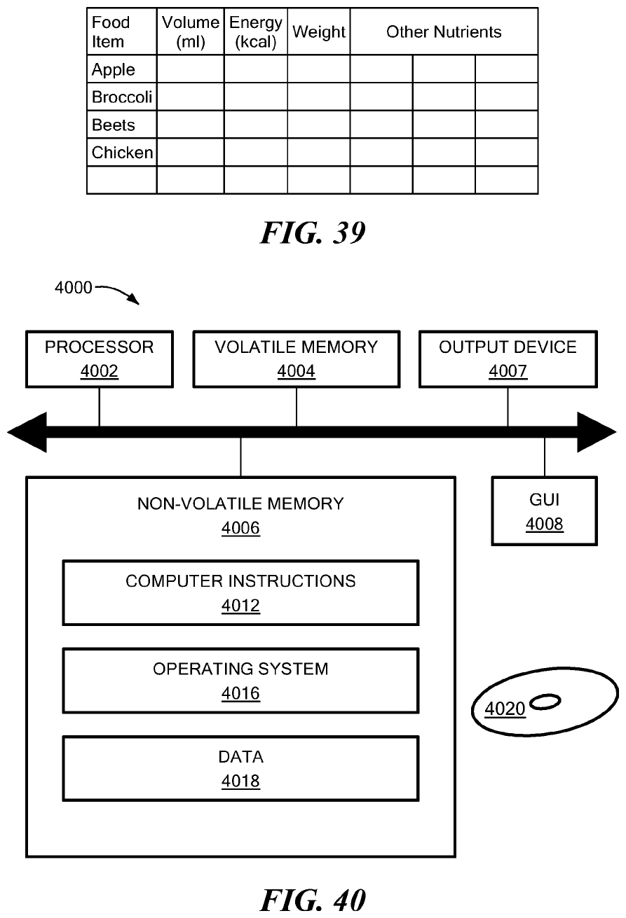

Alternately, a new food database, can be created using the information obtained from the above-generated ground truth. This database comprises of the input MC content, the percentage of food consumed, and the ground truth caloric content. In a few cases, if the database does not have a particular food item, then the user can provide the name/recipe of the food item. This can be used to search the ingredients and determine the amount of ingredients used and their respective calories. This can be seen in FIG. 39.

### Example 1

National Athletic Trainers' Association (NATA) [https://www.nata.org/] provides suggestions for safe weight loss and weight maintenance strategies for all individuals involved in sports and physical activities. These recommendations are based on a preponderance of the scientific evidence that supports safe and effective weight loss and weight management practices and techniques, regardless of the activity or performance goals. However, athletes often do not follow these recommendations and attempt to lose weight by skipping meals, limiting caloric or consuming a specific diet, engaging in pathogenic weight control behaviors, and restricting fluids. Additionally, the pressure of the sport or activity, coaches, peers, or parents drive them to adopt negative body images and unsafe practices to maintain an ideal body composition for the activity. The presented disclosure can aid athletic trainers in providing nutrition information for athletes based on individual needs. Moreover, it can help them to gain knowledge of proper nutrition, weight management practices, and methods to change body composition.

### Example 2

According to the Natural Resources Defense Council (NRDC), approximately 40% of the United States is never eaten, leading to food wastage. According to National Geographic Magazine [National Geographic Society (2014). National Geographic Magazine, Mindsuckers, November 2014, 8. National Geographic Society] an average family of four wastes 1,160 pounds of food annually that is approximately 25% of the food purchased. This costs roughly $1,365 to $2,275 per year [Gunders, Dana. “Wasted: How America is losing up to 40 percent of its food from farm to fork to landfill.” Natural Resources Defense Council 26 (2012)]. The presented disclosure can aid individuals or groups to help plan meals and recommend recipes in advance, thereby reducing the food wastage. By making meal plans, the food items can be bought according to the recipes or serving size.

In cases where food wastage still exists (for example, food from restaurants, fast foods, etc.), the presented disclosure can help the franchise determine the amount of food waste per day and optimize the amount of food cooked. Furthermore, the presented disclosure can also help in the demographic usage of food (for example, in some demographic regions, people may consume only fish). This can help the franchise to concentrate more on the preparing the specific item (according to the example, it is fish) and reduce using other food items.

In some embodiments, digital images can be used to measure food consumption in a restaurant setting. A system can accurately detect, identify, and classify a select set of images from a quick-service restaurant (QSR) and recreate those images using 3-D model reconstruction to detect, identify, and classify, estimate volume and weight from 3-D reconstruction of those foods, and report accurate nutrient intake without relying on human coders. People often do not consume an entire serving, particularly in restaurant settings with large portions, so that measuring actual consumption, not serving sizes, is helpful to understanding dietary intake.

In embodiments, multimedia content can include image and video acquisition from a wide variety of sources using various techniques to generate a large database to train our system. For example, images and/or video of QSR menu items can be taken before, during, and after consumption. In some embodiments, a multi-angle video around the food can be taken aerially for some period of time, such as approximately 15 seconds. A reference marker, such as a blank, white, business-sized card, is added and another video taken. The marker will be removed, and leftover foods will be laid out on grid paper with beverages emptied into a clear pre-marked, scientific plastic cup. A third video will be taken with the amount of leftovers will be varied from 10% to 90%.

It is understood that embodiments of the disclosure are not limited to the particular aspects described. It is also understood that the terminology used herein is for the purpose of describing particular aspects only and is not intended to be limiting. The scope of the claimed invention will be limited only by the claims. As used herein, the singular forms “a”, “an”, and “the” include plural aspects unless the context clearly dictates otherwise.

It should be apparent to those skilled in the art that many additional modifications beside those described are possible without departing from the inventive concepts. all terms should be interpreted in the broadest possible manner consistent with the context in interpreting this disclosure. Variations of the term “comprising”, “including”, or “having” should be interpreted as referring to elements, components, or steps in a non-exclusive manner, so the referenced elements, components, or steps may be combined with other elements, components, or steps that are not expressly referenced. Aspects referenced as “comprising”, “including”, or “having” certain elements are also contemplated as “consisting essentially of” and “consisting of” those elements unless the context clearly dictates otherwise. It should be appreciated that aspects of the disclosure that are described with respect to a system are applicable to the methods, and vice versa, unless the context explicitly dictates otherwise. Furthermore, the word “may” is used throughout this application in a permissive sense (i.e., having the potential to, being able to), not in a mandatory sense (i.e., must).

Aspects of the disclosure that are described with respect to a method are applicable to aspects related to systems and other methods of the disclosure, unless the context clearly dictates otherwise. Similarly, aspects of the disclosure that are described with respect to a system are applicable to aspects related to methods and other systems of the disclosure unless the context clearly dictates otherwise.

In the drawings, similar symbols typically identify similar components unless context dictates otherwise. The numerous innovative teachings of the present disclosure will be described with particular reference to several embodiments (by way of example and not of limitation). It will be readily understood that the aspects of the present disclosure, as generally described herein, and illustrated in the Figures, can be arranged, substituted, combined, separated, and designed in a wide variety of different configurations, all of which are explicitly contemplated herein.

FIG. 40 shows an exemplary computer 4000 that can perform at least part of the processing described herein. The computer 4000 includes a processor 4002, a volatile memory 4004, a non-volatile memory 4006 (e.g., hard disk), an output device 4007 and a graphical user interface (GUI) 4008 (e.g., a mouse, a keyboard, a display, for example). The non-volatile memory 4006 stores computer instructions 4012, an operating system 4016 and data 4018. In one example, the computer instructions 4012 are executed by the processor 4002 out of volatile memory 4004. In one embodiment, an article 4020 comprises non-transitory computer-readable instructions.

Processing may be implemented in hardware, software, or a combination of the two. Processing may be implemented in computer programs executed on programmable computers/machines that each includes a processor, a storage medium or other article of manufacture that is readable by the processor (including volatile and non-volatile memory and/or storage elements), at least one input device, and one or more output devices. Program code may be applied to data entered using an input device to perform processing and to generate output information.

The system can perform processing, at least in part, via a computer program product, (e.g., in a machine-readable storage device), for execution by, or to control the operation of, data processing apparatus (e.g., a programmable processor, a computer, or multiple computers). Each such program may be implemented in a high-level procedural or object-oriented programming language to communicate with a computer system. However, the programs may be implemented in assembly or machine language. The language may be a compiled or an interpreted language and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. A computer program may be stored on a storage medium or device (e.g., RAM/ROM, CD-ROM, hard disk, or magnetic diskette) that is readable by a general or special purpose programmable computer for configuring and operating the computer when the computer reads the storage medium or device.

Processing may also be implemented as a machine-readable storage medium, configured with a computer program, where upon execution, instructions in the computer program cause the computer to operate.

Processing may be performed by one or more programmable processors executing one or more computer programs to perform the functions of the system. All or part of the system may be implemented as, special purpose logic circuitry (e.g., an FPGA (field-programmable gate array), a general-purpose graphical processing units (GPGPU), and/or an ASIC (application-specific integrated circuit)).

Having described exemplary embodiments of the disclosure, it will now become apparent to one of ordinary skill in the art that other embodiments incorporating their concepts may also be used. The embodiments contained herein should not be limited to disclosed embodiments but rather should be limited only by the spirit and scope of the appended claims. All publications and references cited herein are expressly incorporated herein by reference in their entirety.

Elements of different embodiments described herein may be combined to form other embodiments not specifically set forth above. Various elements, which are described in the context of a single embodiment, may also be provided separately or in any suitable subcombination. Other embodiments not specifically described herein are also within the scope of the following claims.

1. An artificial intelligence-based meal planning and recipe recommendation system comprising:
a. providing a plurality of individual's characteristics that represents physical/financial conditions;
b. a database that can store individual profiles comprising recommended/required nutritional/calories intake levels about the user,
c. a meal planning engine configured to use artificial intelligence and/or machine learning-based to generate at least one meal plan based on the individual characteristics and said recommended/required nutritional/calories intake levels; and
d. a recipe recommendation engine configured to use artificial intelligence and/or machine learning-based algorithms to suggest a plurality of recipes for at least one meal plan based on the individual characteristics and said recommended/required nutritional/calories intake levels,
wherein said recommended/required nutritional/calories intake levels comprise at least recommended maximum or minimum macronutrient and/or micronutrients or calories.

2. The system of claim 1 wherein the individual's characteristics comprise personal data, diet goals, food allergies, activities, budget, medical information, sensor information and/or genetic characteristics and are incorporated in the said database.

3. The system of claim 1 wherein the database further includes previous meal plans, current meal plans and their respective calories and said meal planning system generates said at least one meal that satisfies the recommended/required nutritional/calories intake levels about the user.

4. The system of claim 1 further comprising recommending at least one meal and one recipe with complete ingredients from the said database.

5. The system of claim 1 further comprising a genomic database with dietary guidelines for each genotype, the said genomic database configured to recommend at least one meal and/or one recipe with complete ingredients by considering the genetic characteristics.

6. The system of claim 1 further comprising a medical treatment database with dietary guidelines for each medical condition, the said medical treatments database configured to recommend at least one meal and/or one recipe with complete ingredients by considering only the allowed ingredients.

7. The system of claim 1 further comprising a database to store a list entry comprising: an original ingredient of one of the recommended recipes and a corresponding alternative ingredient(s) along with their nutritional values, and wherein the artificial intelligence-based recipe recommendation system substitutes an original ingredient of the selected recipe with the corresponding alternative ingredient for providing an adapted recipe to the individual with allergens information.

8. The system of claim 7 further comprising an artificial intelligence system that can provide alternative ingredient(s) for substituting a standard amount of the original ingredient such that it has a similar effect and approximately the same nutrient value as of the standard amount of the original ingredient.

9. The system of claim 1 further comprising a shopping database configured to save the ingredients purchased and their respective expiration date, and the meal planning and recipe recommendation system configured to adapt to the ingredients available.

10. The system of claim 1 further comprising an artificial intelligence-based shopping list generating system, the said shopping list generating system configured to consider the budget provided by the user and recommend a list of ingredients that can be purchased by the user within that budget.

11. A method of performing hypercomplex convolution, the method comprising the steps of:
a. receiving at least one input multimedia content;
b. placing a kernel window over the pixels in the input multimedia content; and
c. performing hypercomplex convolution with the rest of the pixels and storing the output.

12. The method of claim 11 further comprising performing weight normalization by manipulating the hypercomplex weights to speed up the convergence of the network.

13. The method of claim 12 further comprising performing the weight standardization by computing the mean and standard deviation of the hypercomplex weights and normalizing the weights using these parameters.

14. A method of performing alpha trimmed convolution, the method comprising the steps of:
a. receiving at least one input multimedia content;
b. placing a kernel/window over the pixels in the input multimedia content;
c. sorting the pixels captured in the said kernel;
d. trimming the beginning and the end of the said ordered pixels;
e. performing convolution with remaining ones of the pixels; and
f. storing the output.

15. A method of dietary assessment using multimedia content images, the method comprising the steps of:
a. receiving a plurality of input multimedia content from various positions above food items before and after consumption, including low-resolution input multimedia content;
b. performing super-resolution on the received low-resolution input multimedia content to estimate a high-resolution multimedia content;
c. performing food object detection and localization on at least one input multimedia content;
d. performing three-dimensional multimedia reconstruction using the plurality of input multimedia content;
e. mapping the two-dimensional object localization points to the three-dimensional multimedia content;
f. separating at least one food item detected in the three-dimensional multimedia content;
g. estimating a volume of the at least one food item based on at least the three-dimensional multimedia reconstruction; and
h. mapping the volume to calories using a nutritional database;

16. A method of dietary assessment with deep learning techniques using an acquired multimedia content image, the method comprising the steps of:
a. receiving a plurality of input multimedia content including low-resolution input multimedia content from various positions above food items before and after consumption;
b. performing super-resolution on the received low-resolution input multimedia content to estimate a high-resolution multimedia content;
c. performing food segmentation on at least one input multimedia content;
d. performing depth map estimation on at least one input multimedia content; and
e. performing calorie estimation by providing inputs from the previous steps.

17. The method of claim 16, further comprising, prior to step b), performing a color space transformation on the input multimedia content and selecting a channel from the transformation to create the transformed grayscale channel of the input multimedia content.

18. The method of claim 17, wherein the step of super-resolution using the training dataset and reference dataset further comprises:
a. generating a set of training data;
b. training a hypercomplex/traditional/alpha trimmed convolutional neural network that is parameterized by first weights and biases by comparing one or more characteristics of the training data to one or more characteristics of at least a section of the reference dataset, wherein the network is trained to generate super-resolved image data from low-resolution image data and wherein the training includes modifying one or more of the first weights and biases to optimize processed visual data based on the comparison between the one or more characteristics of the training data and the one or more characteristics of the reference dataset;
c. applying the said trained convolution neural network on low-resolution multimedia content to generate a high-resolution version of the same.

19. The method of claim 18, wherein the convolutional neural network further comprises a plurality of layers among which at least one of the layers applies alpha trimmed convolution, hypercomplex and/or classical convolution layers, and α log and/or α log P activation layers to build a network that can be sequential, recurrent, recursive, branching, or merging.

20. The method of claim 19, wherein the trained convolution neural network is configured to generate a high-resolution version of the input multimedia content by removing artifacts, performing de-mosaicing, and/or de-noising.

21. The method of claim 16, wherein the step of food object detection and localization using classical methods further comprises:
a. receiving a plurality of input multimedia content along with the bounding boxes;
b. training a model by performing feature detection and description on the received multimedia content, building a vocabulary and creating clusters, and feeding it to a classifier engine to classify the objects;
c. testing by receiving a new multimedia content, applying region proposal algorithms on the received content, performing feature detection and description on the said proposed regions, applying the said trained model to classify the objects in the proposed regions, performing proposal reduction to remove overlapping proposals and provide an output which includes a multimedia content with the position of the object and the class of the object.

22. The method of claim 16, wherein the step of food object detection and localization using deep learning methods further comprises:
a. generating a set of training data;
b. training a convolutional neural network that is parameterized by first weights and biases by comparing one or more characteristics of the training data to one or more characteristics of at least a section of the reference dataset, wherein the network is trained to extract high dimensional features that include modifying one or more of the first weights and biases to optimize processed visual data based on the comparison between the one or more characteristics of the training data and the one or more characteristics of the reference dataset;
c. the said training process includes applying regression, anchor, and classification models wherein regression model learns the bounding boxes around the objects, anchors help in detecting the position of the object and classification model learns the class of the object; and
d. testing by receiving new multimedia content, applying the trained convolutional neural network to extract features, the said features are fed to anchors, regression, and classification models to detect objects in the proposed regions, apply proposal reduction to remove overlapping proposals, and provide an output which includes a multimedia content with the position of the object and the class of the object.

23. The method of claim 22, wherein the convolutional neural network further comprises a plurality of layers among which at least one of the layers applies alpha trimmed, hypercomplex convolution and/or classical convolution layers, and α log and/or α log P activation layers to build a network that can be sequential, recurrent, recursive, branching, or merging.

24. The method of claim 23, wherein the training process can be extended to perform semantic segmentation using the features, wherein the said semantic segmentation archives fine-grained inference by making dense predictions inferring labels for every pixel, so that each pixel is labeled with the class of its enclosing object or region.

25. The method of claim 16, wherein three-dimensional multimedia reconstruction comprises:
a. receiving a plurality of input multimedia content taken around the object from a different position;
b. performing image preprocessing wherein preprocessing include denoising, super-resolution, filtering, and/or rectifying pairs of multimedia content;
c, extracting and matching a plurality of features to produce feature correspondences;
d. perform pose estimation and bundle adjustment;
e. obtaining a three-dimensional sparse point cloud;
f. de-noising point cloud;
g. generating three-dimensional sparse point cloud;
h. generating a mesh using the said three-dimensional sparse point cloud; and
i. performing mesh refinement and mapping texture to the mesh.

26. The method of claim 25, wherein input to the three-dimensional multimedia reconstruction may comprise multiple videos obtained from different sensors.

27. The method of claim 26, wherein estimating a pose further comprises:
a. employing the multimedia patch correspondence to generate a plurality of feature tracks;
b. applying global/incremental based methods to determine the three-dimensional points;
c. refining the best pose using an iterative minimization of a robust cost function of re-projection errors to obtain a final pose.

28. The method of claim 16, wherein estimating volume comprises of:
a. receiving a plurality of input multimedia content taken around the object from the different position before and after consumption;
b. performing three-dimensional reconstruction of the food items, thereby having two models (before and after consumption);
c. performing object localization and determining at least one food item on both the models;
d. performing slicing on the said food item from both the models and compute the volume of each slice and sum it up to estimate the complete volume of the said food item; and
e. finding the difference between the said food item before and after consumption to estimate the food intake and the food wasted.

29. The method of claim 16, wherein the step of food segmentation using deep learning methods further comprises:
a. generating a set of training data;
b. training a convolutional neural network that is parameterized by first weights and biases by comparing one or more characteristics of the training data to one or more characteristics of at least a section of the reference dataset, wherein the network is trained to extract high dimensional features that include modifying one or more of the first weights and biases to optimize processed visual data based on the comparison between the one or more characteristics of the training data and the one or more characteristics of the reference dataset;
c. the said training process includes applying regression to detect the items present and providing a specific value for that item; and
d. testing by receiving a new multimedia content, applying the trained convolutional neural network to extract features, the said features are utilized to provide an output comprising a multimedia content with the mask of the object and the class of the object.

30. The method of claim 29, wherein the convolutional neural network further comprises plurality of layers among which at least one of the layers applies alpha trimmed, hypercomplex convolution and/or classical convolution layers, and α log and/or α log P activation layers to build a network that can be sequential, recurrent, recursive, branching, or merging.

31. The method of claim 16, wherein the step of depth map using deep learning methods further comprises:
a. generating a set of training data;
b. training a convolutional neural network that is parameterized by first weights and biases by comparing one or more characteristics of the training data to one or more characteristics of at least a section of the reference dataset, wherein the network is trained to extract high dimensional features that include modifying one or more of the first weights and biases to optimize processed visual data based on the comparison between the one or more characteristics of the training data and the one or more characteristics of the reference dataset;
c. the said training process includes applying regression to estimate the depth of the said multimedia content; and
d. testing by receiving new multimedia content, applying the trained convolutional neural network to extract features, the said features are utilized to provide an output which comprises a multimedia content with the depth map.

32. The method of claim 31, wherein the convolutional neural network further comprises plurality of layers among which at least one of the layers applies alpha trimmed, hypercomplex convolution and/or classical convolution layers, and α log and/or α log P activation layers to build a network that can be sequential, recurrent, recursive, branching, or merging.

33. The method of claim 16, wherein the step of calorie estimation using deep learning methods further comprise of:
a. generating a set of training data;
b. training a convolutional neural network that is parameterized by first weights and biases by comparing one or more characteristics of the training data to one or more characteristics of at least a section of the reference dataset, wherein the network is trained to extract high dimensional features that includes modifying one or more of the first weights and biases to optimize processed visual data based on the comparison between the one or more characteristics of the training data and the one or more characteristics of the reference dataset;
c. the said training process includes applying regression to estimate the calorie content of the said multimedia content; and
d. testing by receiving a new multimedia content, applying the trained convolutional neural network to extract features, the said features are utilized to provide an output which comprises an approximate calorie content of the items in consideration.

34. The method of claim 31, wherein the convolutional neural network further comprises plurality of layers among which at least one of the layers applies alpha trimmed, hypercomplex convolution and/or classical convolution layers, and α log and/or α log P activation layers to build a network that can be sequential, recurrent, recursive, branching, or merging.
